(19) United States
(12) Patent Application Publication
(10) Pub. No.: US 2008/0095750 A1
(43) Pub. Date: Apr. 24, 2008

(54) USE OF ADIPOSE-DERIVED STEM CELLS FOR TREATMENT OF LEUKODYSTROPHIES

(75) Inventors: Jeffrey M. Gimble, Baton Rouge, LA (US); Bruce A. Bunnell, Covington, LA (US); Mandi Lopez, Baton Rouge, LA (US)

Correspondence Address: SEED INTELLECTUAL PROPERTY LAW GROUP PLLC, 701 FIFTH AVE, SUITE 5400, SEATTLE, WA, 98104, US

(73) Assignees: PENNINGTON BIOMEDICAL RESEARCH CENTER, LOUISIANA STATE UNIVERSITY SYSTEM, Baton Rouge, LA (US); TULANE NATIONAL PRIMATE RESEARCH CENTER, Covington, LA (US)

(21) Appl. No.: 11/746,246

(22) Filed: May 9, 2007

Related U.S. Application Data
(60) Provisional application No. 60/799,524, filed on May 10, 2006.

(51) Int. Cl. A61P 25/02; A61K 35/12; C12N 5/08; C12Q 1/54

(52) U.S. Cl. 424/93.7; 435/14; 435/372

(57) ABSTRACT

The present invention relates to a treatment of a leukodystrophy by administration of an adipose-derived stem cell. Specifically, the present invention relates to the treatment of Krabbe disease with an adipose derived stem cell differentiated to express galactocerebrosidase.

### CROSS-REFERENCE TO RELATED APPLICATION

This application claims the benefit under 35 U.S.C. § 119(e) of U.S. Provisional Patent Application No. 60/799,524, filed May 10, 2006, where this provisional application is incorporated herein by reference in its entirety.

### BACKGROUND OF THE INVENTION

Hereditary Metabolic Disorders include the eight identified leukodystrophies: metachromatic leukodystrophy, Refsum's disease, adrenoleukodystrophy, Krabbe disease, phenylketonuria, Canavan disease, Pelizaeus-Merzbacher disease and Alexander's disease. The clinical course of hereditary demyelinating disorders, which usually tend to manifest themselves in infancy or early childhood, is devastating. Previously normal children are deprived, in rapid progression, of sight, hearing, speech, and ambulation. The prognosis is death within a few years.

Krabbe disease, also known as globoid cell leukodystrophy, was first described in humans as an autosomal recessive trait and has subsequently been identified in mice, dogs, cats, sheep, and rhesus monkeys (Baskin, 1989, Lab Invest. 60:7A; Baskin, 1998, Lab Anim. Sci. 48(5):476-482; Suzuki, 1985, Neurochem. Pathol. 3(1):53-68; Wenger, 2000, Mol. Med. Today 6(11):449-451). Krabbe disease is a lysosomal storage disease caused by a mutation in the galactocerebrosidase enzyme which is a lysosomal hydrolase that catabolizes galactosylceramide, a lipid component of myelin. The absence of galactocerebrosidase (GALC) activity results in inadequate myelination and certain morphologic changes that are similar in all species. The histopathologic hallmark of this disease is the appearance of globoid cells in the white matter of the central nervous system located predominantly around blood vessels. Globoid cells are composed of macrophages that have accumulated large amounts of glycolipids in their cytoplasm. In addition to the formation of globoid cells, there is extensive loss of myelin and astrocytosis in the white matter of the central nervous system which affects both the central and peripheral nervous system. In peripheral nerves, axonal degeneration, fibrosis and macrophage infiltration are often present (Suzuki et al., 1983, In: Stanbury J W, J B; Fredrickson, D S; Goldstein, J I; Brown, M S, ed. The Metabolic Basis of Inherited Disease: McGraw Hill 1983:857-880). Analysis by transmission electron microscopy has identified characteristic tubular or crystalline inclusions in the cytoplasm of cells in the brain and kidney (Andrews et al., 1970, Arch Pathol. 89(1):53-55). There are also characteristic changes observable by magnetic resonance imaging (MRI) and computerized tomography (CT) (Baram et al., 1986, Neurology 36(1):111-115; Farley et al., 1992, Pediatr. Neurol. 8(6):455-458; Barone et al., 1996 Am. J. Med. Genet. 63(1):209-217 Percy et al., 1994, Acta. Neuropathol. (Berl) 88(1):26-32; Demaerel et al., 1991, Neuroradiology 33(4):368-371; Sasaki et al., 1991, Pediatr. Neurol. 7(4):283-288; Zafeiriou et al., 1996, Pediatr. Neurol. 15(3):240-244). Typical MRI findings in humans with Krabbe disease include central and cortical atrophy, ventricular dilatation, decreased white matter volume, and focal dense lesions. Although results vary between cases, MRI is a highly effective technique to map lesions and to follow disease progression during life.

One of the most important and unique features of Krabbe disease is the elevation in the white matter of psychosine (galactosylsphingosine) (Kobayashi et al., 1988, Ann. Neurol. 24(4):517-522). Psychosine is normally formed in oligodendroglia during the period of active myelination by the addition of galactose to sphingosine and is rapidly turned over in normal individuals (Svennerholm et al., 1980, J. Lipid Res. 21(1):53-64); however, psychosine degradation is impaired in patients with Krabbe disease where their brains contain 10 to 100 times the normal amount of this lipid (Wenger, 2000, Mol. Med. Today 6(11):449-451; Miyataki et al., 1972, BBRC. 48:538-543; Svennerholm et al., 1980, J. Lipid Res. 21(1):53-64; Wenger, 2000, Mol. Med. Today 6(11):449-451; Vanier et al., 1976, Adv. Exp. Med. Biol. 68:115-126). Even though psychosine accounts for less than 0.1% of the galactosylceramide in the white matter of Krabbe patients, it is apparently cytotoxic (Suzuki et al., 1976, In: Volk B S, L, ed. Current Trends in Sphingolipidosis and Allied Disorders. New York: Plenum Press; Taketomi et al., 1964, Jpn. J. Exp. Med. 34:255-265). As psychosine accumulates, myelin formation ceases prematurely as the oligodendroglia are destroyed (Wenger, 2000, Mol. Med. Today 6(11):449-451). Thus, the lack of GALC activity induces the primary features of Krabbe disease including loss of myelin, loss of oligodendroglia, formation of globoid cells, and the production of psychosine without massive accumulation of the substrate for GALC, galactosylceramide.

Most clinical cases of Krabbe disease manifest during infancy and progress rapidly to death during childhood. Human infants affected by Krabbe disease exhibit a variety of behavioral signs, including irritability, excessive crying, loss of motor skills, hypersensitivity to external stimuli, stiffness of muscles, extension of arms and legs, clenched fingers, hypotonicity, blindness, and deafness (Suzuki, 1985, Neurochem. Pathol. 3(1):53-68; Gullotta et al., 1979, Neuropadiatrie. 10(4):395-400; D'Angostino et al., 1963, Arch Neurol. 8:82-112; Hagberg et al., 1963, J. Neurol. Neurosurg. Psychiatry. 26:195-198; Suzuki et al., 1983, In: Stanbury J W, J B; Fredrickson, D S; Goldstein, J I; Brown, M S, ed. The Metabolic Basis of Inherited Disease: McGraw Hill 1983:857-880). There is phenotypic variability in the age of onset and clinical signs in infants affected with globoid cell-like leukodystrophies like Krabbe disease. Clinical signs in human infants with Krabbe disease include growth arrest, progressive microcephaly, and severe failure to thrive (Zlotogora et al., 1986, Acta. Paediatr. Scand. 75(2):251-254). Affected individuals can be definitively diagnosed by demonstrating deficient GALC activity in leukocytes or cultured skin fibroblasts. Prenatal diagnosis can be made using chorionic villus samples or cultured amniotic fluid cells. The diagnosis of carriers is more problematic because obligate heterozygotes have a wide range of enzymatic activity that overlaps that of unrelated normal individuals (Wenger et al., 1993, Boston: Butterworth-Heinemann; Wenger et al., 1991, New York: Wiley-Liss).

To date, treatment options for Krabbe disease are limited. Enzyme replacement therapy can reduce the rate of disease progression but does not prevent death at an early age. Transplantation of bone marrow or umbilical cord cells, including hematopoietic and mesenchymal stem cells, can reverse disease progression but such transplantations are often complicated by the significant consequences of graft versus host disease. It has recently been demonstrated that human adipose tissue is a rich source of stromal-like adult stem cells and based on the original methods described by Hauner and others, reproducible and efficient methods have been developed to isolate adult stem cells from human liposuction tissue (Hauner et al., 1989, J. Clin. Invest. 84(5):1663-1670; Hauner et al., 1988, Horm. Metab. Res. Suppl. 19:35-39; Gimble et al., 2003, Curr. Top Dev. Biol. 58:137-160; Aust et al., 2004, Cytotherapy 6(1):7-14; Awad et al., 2003, Tissue Eng. 9(6):1301-1312; Awad et al., 2004, Biomaterials 25(16):3211-3222; Elmslie et al., 2000, J. Clin. Psychiatry 61(3):179-184; Delany et al., 2005, Mol. Cell Proteomics 4:731-740; Gronthos et al., 2001, J. Cell Physiol. 189(1):54-63; Halvorsen et al., 2000, Int. J. Obes. Relat. Metab. Disord. 24 Suppl. 4:S41-44; Halvorsen et al., 2001, Metabolism 50(4):407-413; Halvorsen et al., 2001, Tissue Eng. 7(6):729-741; Hicok et al., 2004, Tissue Eng. 10(3-4):371-380; Safford et al., 2002, Biochem. Biophys. Res. Commun. 294(2):371-379; Safford et al., 2004, Exp. Neurol. 187(2):319-328; Sen et al., 2001, J. Cell Biochem. 81(2):312-319; Wickham et al., 2003, Clin. Orthop. (412):196-212; Guilak et al., 2005, J. Cell Physiol.; Mitchell et al., Stem Cells online Jan. 12, 2006: 2005-0235; Aust et al., 2004, Cytotherapy 6(1):7-14; Halvorsen et al., 2001, Metabolism 50(4):407-413. Thus, adipose-derived adult stem cells (ASCs) offer an alternative in vitro model for the treatment of leukodystrophies such as Krabbe disease (Gimble, 2003, Expert Opinion in Biological Therapy 3: 705-713; Gimble and Guilak, 2003, Current Topics in Developmental Biology, 58: 137-160) as they are readily available, abundant, and are incapable of generating a graft versus host immune reaction.

ASCs can be reproducibly isolated from liposuction aspirates through a procedure involving collagenase digestion, differential centrifugation, and expansion in culture such that a single milliliter of tissue yields over 400,000 cells (Aust, et al., 2004, Cytotherapy 6: 1-8). Undifferentiated human adipocyte cells express a distinct immunophenotype based on flow cytometric analyses and, following induction, produce additional adipocyte specific proteins (Aust, et al., 2004, Cytotherapy 6: 1-8; 2001, J. Cell Physiol., 189: 54-63; Halvorsen, et al., 2001, Metabolism 50: 407-413; Sen, 2001, J. Cell. Biochem. 81: 312-319; Zuk, et al., 2002, Mol. Biol. Cell. 13: 4279-4295). Human adipose-derived adult stem cells (huASCs) display multipotentiality, with the capability of differentiating along the adipocyte, chondrocyte, myogenic, neuronal, and osteoblast lineages Aust, et al., 2004, Cytotherapy 6: 1-8; 2001, J. Cell Physiol., 189: 54-63; Halvorsen, et al., 2001, Metabolism 50: 407-413; Sen, 2001, J. Cell. Biochem. 81: 312-319; Zuk, et al., 2002, Mol. Biol. Cell. 13: 4279-4295; Ashjian, et al., 2003, Plast. Reconstr. Surg., 111: 1922-19231; Awad, et al., 2003, Tissue Engineering, 9: 1301-1312; Awad, et al., 2004, Biomaterials 25: 3211-3222; Halvorsen, et al., 2001, Tissue Eng., 7: 729-741; Hicok, et al., 2004, Tissue Engineering 10: 371-380; Mizuno, et al., 2002, Plast. Reconstr. Surg. 109: 199-209; Safford, et al., 2002, Biochem. Biophys. Res. Commun., 294: 371-379; Safford, et al., 2004, Experimental Neurology, 187: 319-328; Wickham, et al., 2003, Clin. Orthop., 412: 196-212; Winter, et al., 2003, Arthritis Rheum., 48: 418-429; Zuk, et al., 2001, Tissue Eng. 7: 211-28). In the presence of dexamethasone, insulin, isobutylmethylxanthine and a thiazolidinedione, the undifferentiated human adipocyte cells undergo adipogenesis as evidenced by the fact that between 30% to 80% of the cells, based on flow cytometric methods, accumulate lipid vacuoles, which can be stained for neutral lipid with Oil Red O dye (Halvorsen, et al., 2001, Metabolism 50: 407-413; Sen, et al., 2001, J. Cell. Biochem., 81: 312-319).

There remains a need in the art for methods of identifying and characterizing differentiated ASCs. The present invention fulfills this need by providing a means for identifying and characterizing ASCs that express GALC which are useful in treating Krabbe disease.

### SUMMARY OF THE INVENTION

The present invention encompasses a method of treating at least one symptom of a leukodystrophy in a mammal. Preferably, the mammal is a primate. More preferably, the mammal is a monkey. Most preferably, the mammal is a human. The method comprises administering to a mammal an isolated adipose-derived stem cell (ASC) exhibiting a non-immunogenic characteristic. Preferably, the ASC expresses galactocerebrosidase.

In one aspect, leukodystrophy is selected from the group consisting of Krabbe disease, adrenoleukodystrophy/adrenomyeloneuropathy, Aicardi-Goutieres syndrome, Alexanders disease, childhood ataxia with diffuse central nervous system hypomyelination (CACH), cerebral autosomal dominant arteriopathy with subcortical infarcts and leukoencephalopathy (CADASIL), Canavan disease, cerebrotendinous xanthomatosis, metachromatic leukodystrophy, neonatal adrenoleukodystrophy, ovarioleukodystrophy syndrome, Pelizaeus-Merzbacher disease, Refsum disease, Van der Knaap syndrome and Zellweger syndrome. Preferably, leukodystrophy is Krabbe disease.

In another aspect, galactocerebrosidase is expressed from ASCs in an amount effective to reduce levels of psychosine in white matter of a brain of a mammal.

In yet another aspect, galactocerebrosidase is expressed from a differentiated ASC in an amount effective to reduce levels of psychosine in white matter of a brain of a mammal.

The invention also includes a method of treating at least one symptom of leukodystrophy, wherein the symptom is selected from the group consisting of axonal degeneration, fibrosis, macrophage infiltration, astrocytosis, decrease in myelin, irritability, excessive crying, loss of motor skills, hypersensitivity to external stimuli, stiffness of muscles, extension of arms and legs, clenched fingers, hypotonicity, blindness and deafness.

In one aspect, the ASC is administered intravenously to the mammal. The ASC can be allogenic or autologous with respect to the mammal.

In a further aspect, the ASC further comprises a biocompatible matrix. The biocompatible matrix is selected from the group consisting of calcium alginate, agarose, fibrin, collagen, laminin, fibronectin, glycosaminoglycan, hyaluronic acid, heparin sulfate, chondroitin sulfate A, dermatan sulfate, and bone matrix gelatin.

In one aspect, the ASCs are cultured in vitro for a period of time without being induced to differentiate prior to being administered to a mammal.

The invention also includes a method of identifying an ASC that expresses galactocerebrosidase in a population of cells derived from adipose tissue. The method comprises providing a substrate specific for galactocerebrosidase to the population of cells, wherein the substrate is degraded when galactocerebrosidase is present in the ASC, thereby identifying an ASC in the population of cells.

In one aspect, the substrate is galactosylsphingosine or galactosylceramide.

In another aspect, an ASC is differentiated into a cell exhibiting at least one characteristic of a cell selected from the group consisting of a leukocyte, a fibroblast, a chondrocyte, an osteoblast, a Schwann cell, an oligodendrocyte and a neuron.

The invention also includes a method of increasing the level of galactocerebrosidase in a tissue or mammal. The method comprises administering an isolated ASC exhibiting a non-immunogenic characteristic to a mammal, wherein the ASC differentiates in vivo or in vitro into a cell that expresses galactocerebrosidase.

In a further aspect, the ASC is differentiated into a cell that exhibits at least one characteristic of a cell selected from the group consisting of a leukocyte, a fibroblast, a chondrocyte, an osteoblast, a Schwann cell, an oligodenderocyte and a neuron.

The invention also includes an isolated ASC exhibiting a non-immunogenic characteristic, wherein the ASC expresses galactocerebrosidase and is identified by providing a substrate specific for galactocerebrosidase to a population of cells, wherein the substrate is degraded when galactocerebrosidase is present in the ASC, thereby identifying an ASC in a population of cells. Preferably, isolated ASC is a human cell.

In another aspect, the invention provides a substantially homogeneous population of isolated ASCs, wherein the isolated ASCs exhibit a non-immunogenic characteristic. In one aspect, the ASCs express galactocerebrosidase and is identified by providing a substrate specific for galactocerebrosidase to the population of cells, wherein the substrate is degraded when galactocerebrosidase is present in the ASC, thereby identifying an ASC in a population of cells.

In a further aspect, the isolated ASCs are genetically modified to express galactocerebrosidase. In yet a further aspect, the isolated ASC is transfected with a vector expressing galactocerebrosidase. In some aspects, the galactocerebrosidase is derived from a human, monkey, mouse or rat.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A and 1B, illustrates adipogenesis in confluent stromal cell cultures that were induced for 3 days with dexamethasone, insulin, isobutylmethylxanthine and a thiazolidinedione followed by culture in the presence of dexamethasone and insulin. After 14 days in culture, the cells were fixed and stained for neutral lipid with Oil Red O (FIG. 1A) and the conditioned medium was assayed for leptin levels by ELISA days (FIG. 1B).

FIG. 2, comprising FIGS. 2A through 2G, is a series of images depicting the morphology, proliferation and differentiation potential of primate adipose stem cells (prASCs). FIGS. 2A and 2B are images representing low density and high density cultures of expanded non-human primate ASCs, respectively, which show the spindle-shaped fibroblastic morphology. FIG. 2C is an image illustrating that primate bone marrow stem cells (prBMSCs) are more heterogeneous compared to pASCs that have fibroblastic morphology. FIG. 2D is an image depicting a single cell that can be expanded into a clonal population and can generate colony forming units (CFUs) as demonstrated by Giemsa staining. FIG. 2E is an image illustrating that passage 3-4 pATSCs retain multilineage differentiation capability undergoing adipogenesis. FIG. 2F is an image illustrating that prATSCs that are at passage 3-4 retain multilineage differentiation capability undergoing osteogenesis. FIG. 2G is an image illustrating that passage 3-4 prATSCs retain multilineage differentiation capability undergoing chondrogenesis.

FIG. 3 illustrates a two-dimensional polyacrylamide gel electrophoresis that was performed with protein lysates prepared from human ADAS cells in undifferentiated (Undiff) and adipocyte differentiated (Diff) condition 9 days following induction. The gels were stained with Sypro Ruby. The figure displays representative gels from each condition as well as the master composite prepared based on features conserved on replicate gels prepared from protein extracts obtained from the four individual donors.

FIG. 4 is a series of images illustrating the individual protein features from undifferentiated (U) and adipocyte differentiated (D) huASCs. Differentiation-dependent changes are identified by arrows marking fatty acid binding protein, adipocyte (3101), HSP20-like protein (7204), stathmin (3107), and elfin/PDZ and Lim domain protein 1 (6521) on a 2D-PAGE analysis of total huASC lysates.

FIG. 5 is a graph representing the weight gain of affected infants compared to the mean and standard deviation of normal male and female infants (20 total animals).

FIG. 6, comprising FIGS. 6A and 6B are two graphs illustrating the neonatal behavioral assessment factor scores at 30-days for affected infants compared to normal infants (FIG. 6A) and 30-day neonatal behavioral assessment neuromotor item scores for affected infants compared to the mean and standard deviation for normal infants (FIG. 6A).

FIG. 7 is a graph illustrating the mean and standard deviation conduction velocities in the Ulnar Nerve by group and age.

### DETAILED DESCRIPTION OF THE INVENTION

ASCs have a vast potential in transplantation and in the treatment of disease. The present invention provides methods and compositions for ASCs differentiated to express at least one characteristic of a non-adipose tissue derived cell. In one embodiment, the ASCs are differentiated into cells that express galactocerebrosidase. The present invention further comprises methods for identifying a differentiated adipose-derived stem cell that expresses galactocerebrosidase in a population of cells derived from adipose tissue. The invention further provides methods of treating leukodystrophies in a mammal by administering an isolated adipose-derived stem cell. Preferably, the mammal is a human. Preferably, the leukodystrophy is Krabbe disease.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Laboratory Press, Cold Spring Harbor, N.Y., and Ausubel et al., 2002, Current Protocols in Molecular Biology, John Wiley & Sons, New York, N.Y.), which are provided throughout this document.

The present abbreviations are used throughout this application.

ASC, Adipose-derived Adult Stem Cell; BMI, Body Mass Index; 2D-PAGE, 2 Dimensional Polyacrylamide Gel Electrophoresis; D, Differentiated; DMEM, Dulbecco's Modified Eagles Medium; hu, human; PBS, Phosphate Buffered Saline; pr, primate; U, Undifferentiated

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent based on the context in which it is used.

The term “adipose tissue-derived cell” refers to a cell that originates from adipose tissue. The initial cell population isolated from adipose tissue is a heterogeneous cell population including, but not limited to stromal vascular fraction (SVF) cells.

“Adipose” refers to any fat tissue. The adipose tissue may be brown or white adipose tissue. Preferably, the adipose tissue is subcutaneous white adipose tissue. The adipose tissue may be from any organism having fat tissue. Preferably the adipose tissue is mammalian, most preferably the adipose tissue is human. A convenient source of human adipose tissue is that derived from liposuction surgery. However, the source of adipose tissue or the method of isolation of adipose tissue is not critical to the invention.

As used herein, the term “adipose-derived adult stem cell (ASC)” refers to stromal cells that originate from adipose tissue which can serve as stem cell-like precursors to a variety of different cell types such as but not limited to adipocytes, osteocytes, chondrocytes, muscle and neuronal/glial cell lineages. Adipose-derived adult stem cells make up a subset population derived from adipose tissue which can be separated from other components of the adipose tissue using standard culturing procedures or other methods disclosed herein. In addition, adipose-derived adult stem cells can be isolated from a mixture of cells using the cell surface markers disclosed herein.

As used herein, the term “adipose cell” is used to refer to any type of adipose tissue, including an undifferentiated adipose-derived adult stem cell and a differentiated adipose-derived adult stem cell.

As used herein, the term “allogeneic” is meant to refer to any material derived from a different mammal of the same species.

As used herein, the term “autologous” is meant to refer to any material derived from the same individual to which it is later to be re-introduced.

As used herein, the term “phenotypic characteristics” should be construed to mean at least one of the following characteristics: morphological appearance, the expression of a specific protein, a staining pattern or the ability to be stained with a substance.

By the term “applicator,” as the term is used herein, is meant any device including, but not limited to, a hypodermic syringe, a pipette, and the like, for administering the compounds and compositions of the invention.

As used herein, “central nervous system” should be construed to include brain and/or the spinal cord of a mammal. The term may also include the eye and optic nerve in some instances.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiation-associated proteins in that cell. For example expression of GALC in a leukocyte is a typical example of a terminally differentiated leukocyte.

“Differentiation medium” is used herein to refer to a cell growth medium comprising an additive or a lack of an additive such that a stem cell, adipose tissue derived stromal cell, embryonic stem cell, ES-like cell, MSCs, neurosphere, NSC or other such progenitor cell, that is not fully differentiated when incubated in the medium, develops into a cell with some or all of the characteristics of a differentiated cell.

When a cell is said to be “differentiating,” as that term is used herein, the cell is in the process of being differentiated.

A “differentiated adipose-derived adult stem cell” is an adipose-derived adult stem cell isolated from any adipose tissue that has differentiated as defined herein.

An “undifferentiated adipose-derived adult stem cell” is a cell isolated from adipose tissue and cultured to promote proliferation, but has no detectably expressed proteins or other phenotypic characteristics indicative of biological specialization and/or adaptation.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “disease, disorder or condition of the central nervous system” is meant to refer to a disease, disorder or a condition which is caused by a genetic mutation in a gene that is expressed by cells of the central nervous system or cells that affect the central nervous system such that one of the effects of such a mutation is manifested by abnormal structure and/or function of the central nervous system, such as, for example, defective myelin. Such genetic defects may be the result of a mutated, non-functional or under-expressed gene in a cell of the central nervous system.

As used herein “endogenous” refers to any material from or produced inside an organism, cell or system.

“Exogenous” refers to any material introduced from or produced outside an organism, cell, or system.

An “isolated cell” refers to a cell which has been separated from other components and/or cells which naturally accompany the isolated cell in a tissue or mammal.

As used herein, a “graft” refers to a cell, tissue or organ that is implanted into an individual, typically to replace, correct or otherwise overcome a defect. A graft may further comprise a scaffold. The tissue or organ may consist of cells that originate from the same individual; this graft is referred to herein by the following interchangeable terms: “autograft”, “autologous transplant”, “autologous implant” and “autologous graft”. A graft comprising cells from a genetically different individual of the same species is referred to herein by the following interchangeable terms: “allograft”, “allogeneic transplant”, “allogeneic implant” and “allogeneic graft”. A graft from an individual to his identical twin is referred to herein as an “isograft”, a “syngeneic transplant”, a “syngeneic implant” or a “syngeneic graft”. A “xenograft”, “xenogeneic transplant” or “xenogeneic implant” refers to a graft from one individual to another of a different species.

“Immunophenotype” of a cell is used herein to refer to the phenotype of a cell in terms of the surface protein profile of a cell.

As used herein, the term “leukodystrophy” refers to a disease or disorder that is characterized by a progressive degeneration of the white matter of the brain due to imperfect growth or development of the myelin sheath, the fatty covering that acts as an insulator around nerve fiber. The “leukodystrophies” are a group of disorders that are caused by genetic defects in how myelin produces or metabolizes one of its chemical constituents. Each of the leukodystrophies is the result of a defect in the gene that controls one of the chemicals. Specific leukodystrophies include but are not limited to Krabbe disease, adrenoleukodystrophy/adrenomyeloneuropathy, Aicardi-Goutieres syndrome, Alexanders disease, childhood ataxia with diffuse central nervous system hypomyelination (CACH), cerebral autosomal dominant arteriopathy with subcortical infarcts and leukoencephalopathy (CADASIL), Canavan disease, cerebrotendinous xanthomatosis, metachromatic leukodystrophy, neonatal adrenoleukodystrophy, ovarioleukodystrophy syndrome, Pelizaeus-Merzbacher disease, Refsum disease, Van der Knaap syndrome and Zellweger syndrome.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The terms “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew itself or to produce progeny cells which will differentiate into the desired cell type. In contrast to pluripotent stem cells, lineage-committed progenitor cells are generally considered to be incapable of giving rise to numerous cell types that phenotypically differ from each other. Instead, progenitor cells give rise to one or possibly two lineage-committed cell types.

As used herein, the term “multipotential” or “multipotentiality” is meant to refer to the capability of a stem cell to differentiate into more than one type of cell.

As used herein, the term “late passaged adipose tissue-derived stromal cell,” refers to a cell exhibiting a less immunogenic characteristic when compared to an earlier passaged cell. The immunogenicity of an adipose tissue-derived stromal cell corresponds to the number of passages. Preferably, the cell has been passaged up to at least the second passage, more preferably, the cell has been passaged up to at least the third passage, and most preferably, the cell has been passaged up to at least the fourth passage.

The term “protein” typically refers to large polypeptides.

The term “peptide” typically refers to short polypeptides.

A “therapeutic” treatment is a treatment administered to a patient who exhibits signs of pathology for the purpose of diminishing or eliminating those signs and/or decreasing or diminishing the frequency, duration and intensity of the signs.

A “therapeutically effective amount” of a compound is that amount of compound which is sufficient to provide a beneficial effect to the subject to which the compound is administered. Also, as used herein, a “therapeutically effective amount” is the amount of cells which is sufficient to provide a beneficial effect to the subject to which the cells are administered.

To “treat” a disease as the term is used herein, means to reduce the frequency of the disease or disorder reducing the frequency with which a symptom of the one or more symptoms disease or disorder is experienced by an animal.

“Xenogeneic” refers to any material derived from a mammal of a different species.

Description

The invention relates to the discovery that ASCs can be utilized to treat leukodystrophies, preferably Krabbe Disease. The present invention also relates to the discovery that adipose-derived stem cells can be differentiated into cells that express galactocerebrosidase (GALC). The ASCs may be characterized in vitro and in vivo in a variety of animal model systems, including but not limited to monkey and canine, for their ability to treat Krabbe disease. The present invention also facilitates the identification of such GALC expressing cells from a heterogeneous differentiated, undifferentiated, or a mixed population of adipose cells. A GALC expressing cell includes but is not limited to a leukocyte, a fibroblast, a chondrocyte, an osteoblast, a Schwann cell, an oligodendrocyte or a neuron. Among the advantages of using ASCs for this purpose is that they are abundant, readily available and incapable of generating a graft versus host immune reaction. The subject may be a mammal, but is preferably a human or a monkey.

I. Methods of Isolating and Differentiating Adipose Stem Cells (ASCs)

In one aspect, the methods of the instant invention can be practiced using an ASC from any animal, preferably a human or monkey. In one embodiment, the monkey is a rhesus monkey. The ASCs may be isolated by a variety of methods known to those skilled in the art. For example, such methods are described in U.S. Pat. No. 6,153,432 incorporated herein in its entirety. In a preferred method, adipose tissue is isolated from a mammalian subject, preferably a human subject. A preferred source of adipose tissue is omental adipose. In humans, the adipose tissue is typically isolated by liposuction. If the cells of the invention are to be transplanted into a human subject, it is preferable that the adipose tissue be isolated from that same subject so as to provide for an autologous transplant. Alternatively, the administered tissue may be allogenic.

In one method of isolating ASCs, the adipose tissue is treated with collagenase at concentrations between 0.01 to 0.5%, preferably 0.04 to 0.2%, most preferably about 0.1%, trypsin at concentrations between 0.01 to 0.5%, preferably 0.04%, most preferably about 0.2%; and/or dispase at concentrations of 0.5 ng/ml to 10 ng/ml; and/or effective concentrations of hyaluronidase or DNase; and ethylenediaminetetra-acetic acid (EDTA) at concentrations of about 0.01 to 2.0 mM, preferably at about 0.1 to about 1.0 mM, most preferably at 0.53 mM; at temperatures between 25° C. to 50° C., preferably between 33° C. to 40° C., most preferably at 37° C., for periods of between 10 minutes to 3 hours, preferably between 30 minutes to 1 hour, most preferably 45 minutes. The cells are passed through a nylon or cheesecloth mesh filter of between 20 microns to 800 microns, more preferably between 40 to 400 microns, most preferably 70 microns. The cells are then subjected to differential centrifugation directly in media or over a Ficoll or Percoll or other particulate gradient. Cells are centrifuged at speeds of between 100 to 3000×g, more preferably 200 to 1500×g, most preferably at 500×g for periods of between 1 minutes to 1 hour, more preferably 2 to 15 minutes, most preferably 5 minutes, at temperatures of from 4° C. to 50° C., preferably from 20° C. to 40° C., most preferably at about 25° C.

Following isolation, ASCs are incubated in stromal cell medium in a culture apparatus for a period of time or until the cells reach confluency before passing the cells to another culture apparatus. The culturing apparatus can be of any culture apparatus commonly used in culturing cells in vitro. Preferably, the level of confluence is greater than 70% before passing the cells to another culture apparatus. More preferably, the level of confluence is greater than 90%. A period of time can be any time suitable for the culture of cells in vitro. Stromal cell medium may be replaced during the culture of the ASCs at any time. Preferably, the stromal cell medium is replaced every 3 to 4 days. ASCs are then harvested from the culture apparatus whereupon they can be used immediately or cryopreserved to be stored for use at a later time. ASCs may be harvested by trypsinization, EDTA treatment, or any other procedure used to harvest cells from a culture apparatus.

Various terms are used to describe cells in culture. Cell culture refers generally to cells taken from a living organism and then grown under controlled conditions. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time. Each round of subculturing is referred to as a passage. Thus, when cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, and time between passaging.

In another aspect, the invention provides an isolated ASC that exhibits a non-immunogenic characteristic and expresses galactocerebrosidase (GALC). Thus, the invention encompasses methods of treating ASCs to induce them to differentiate into a cell that expresses GALC. In one embodiment, the GALC expressing cell is a leukocyte, a fibroblast, a chondrocyte, an osteoblast, a Schwann cell, an oligodendrocyte or a neuron. In one embodiment, the ASC further comprises a biocompatible matrix. Preferably the biocompatible matrix is calcium alginate, agarose, fibrin, collagen, laminin, fibronectin, glycosaminoglycan, hyaluronic acid, heparin sulfate, chondroitin sulfate A, dermatan sulfate or bone matrix gelatin.

While the invention is not bound by any theory of operation, it is believed that treatment of the preadipocytes with a medium containing a combination of serum, embryonic extracts, preferably a non-human embryonic extract, purified or recombinant growth factors, cytokines, hormones, and/or chemical agents, in a 2-dimensional or 3-dimensional microenvironment, will induce differentiation.

The immunophenotype of ASCs changes progressively, depending on culturing procedures (i.e. passage number). The adherence to plastic and subsequent expansion of human ASCs selects for a relatively homogeneous cell population, enriching for cells expressing a “stromal” immunophenotype, as compared to the heterogeneity of the crude stromal vascular fraction. ASCs also express stem-cell associated markers including, but not limited to, human multidrug transporter (ABCG2) and aldehyde dehydrogenase (ALDH).

The immunophenotype of ASCs can be exploited to serve as unique identifiers for ASCs. That is, the unique cell surface markers on the cells of interest can be used to isolate a specific sub-population of cells from a mixed population of cells derived from adipose tissue. Cell surface markers for BMSCs have been characterized (e.g. Meinel et al., 2004, Ann. Biomed. Eng. 32:112-122 and Meinel et al., 2004, J Biomed Mater Res. A. 71:25-35-4, each incorporated herein it its entirety). As exemplified in the present invention, huASCs exhibit a differentiation potential comparable to BMSCs, including expression of the following cell surface markers: CD9, CD10, CD13, CD29, CD44, CD49d, CD54, CD55, CD59, CD71, CD73, CD90, CD105, CD106, CD146, CD166, α-smooth muscle actin, collagen type 1, collagen type III, HLA-ABC, nestin, osteopontin, osteonectin and vimentin. One skilled in the art would appreciate that an antibody specific for a cell surface marker can be conjugated to a physical support (i.e. a streptavidin bead) and therefore be used to bind and isolate ASCs having that specific cell surface marker. An example of an antibody that specifically binds to an ASC includes, but is not limited to, anti-ABCG2 antibody. After binding, the bound ASCs can be separated from the remaining cells by, for instance, magnetic separation using magnetic beads, including but not limited to Dynabeads® (Dynal Biotech, Brown Deer, Wis.). Further to the use of Dynabeads®, MACS separation reagents (Miltenyi Biotec, Auburn, Calif.) can be used to remove ASCs from a mixed population of cells. Alternatively, the immunophenotype of ASCs permits sorting using a flow cytometry-based cell sorter. As a result of the separation step or cell sorting, a population of enriched ASCs or enriched can be obtained. Preferably, the population of ASCs is a purified cell population. The isolated ASCs can then be cultured and expanded in vitro using methods disclosed herein or conventional methods.

Non-limiting examples of base media useful in the methods of the invention include Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM—without serum), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. A preferred medium for use in the present invention is DMEM. These and other useful media are available from GIBCO, Grand Island, N.Y., USA and Biological Industries, Bet HaEmek, Israel, among others. A number of these media are summarized in Methods in Enzymology, Volume LVIII, “Cell Culture”, pp. 62-72, edited by William B. Jakoby and Ira H. Pastan, published by Academic Press, Inc.

Additional non-limiting examples of media useful in the methods of the invention can contain fetal serum of bovine or other species at a concentration of at least 1% to about 30%, preferably at least about 5% to 15%, mostly preferably about 10%. Embryonic extract of chicken or other species can be present at a concentration of about 1% to 30%, preferably at least about 5% to 15%, most preferably about 10%.

By “growth factors, cytokines, hormones” refers to the following specific factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor, fibroblast growth factor, nerve growth factor, cilary neurotrophic factor, platelet derived growth factor, and bone morphogenetic protein at concentrations of between picogram/ml to milligram/ml levels. At such concentrations, the growth factors, cytokines and hormones useful in the methods of the invention are able to induce, up to 100% the formation of blood cells (lymphoid, erythroid, myeloid or platelet lineages) from adipose derived stromal cells in colony forming unit (CFU) assays. (Moore et al., 1973, J. Natl. Cancer Inst. 50:603-623; Lee et al., 1989, J. Immunol. 142:3875-3883; Medina et al., 1993, J. Exp. Med. 178:1507-1515).

It is further recognized that additional components may be added to the culture medium. Such components may be antibiotics, antimycotics, albumin, amino acids, and other components known to the art for the culture of cells. Additionally, components may be added to enhance the differentiation process, for example to enhance differentiation into a cell that expresses GALC. By “chemical agents” is meant to include, but not be limited to, antioxidant compounds such as butylated hydroxyanisole (BHA) or 2-mercaptoethanol, steroids, retinoids, and other chemical compounds or agents that induce the differentiation of ASCs. In one embodiment, the ASCs are cultured in insulin, dexamethasone and isobutylmethylxanthine.

In another aspect, the invention provides a method of identifying and/or characterizing an ASC that expresses GALC in a population of cells derived from adipose tissue. The ASCs may be characterized for efficacy by one or more of the methods discussed herein in one of the readily available animal models for Krabbe disease, including but not limited to a mouse model, a canine model or a monkey model. Preferably the monkey is a rhesus monkey.

“Characterization” of the resulting differentiated cells is intended to refer to the identification of surface and intracellular proteins, genes, and/or other markers indicative of the lineage commitment of the ASCs to a particular terminal differentiated state. These methods will include, but are not limited to (a) detection of cell surface proteins by immunofluorescent methods using protein specific monoclonal antibodies linked using a secondary fluorescent tag, including the use of flow cytometric methods; (b) detection of intracellular proteins by immunofluorescent methods using protein specific monoclonal antibodies linked using a secondary fluorescent tag, including the use of flow cytometric methods; (c) detection of cell genes by polymerase chain reaction, in situ hybridization, and/or northern blot analysis; and/or (d) detection of GALC expression; (e) detection of GALC activity.

In a preferred embodiment, the method of identifying a GALC expressing cell comprises providing a substrate specific for GALC to the population of cells and wherein the substrate is degraded when present in the ASC thereby identifying the ASC in the population of cells. In one embodiment, the substrate is galactosylsphingosine or galactosylceramide.

Partially or terminally differentiated cells may be characterized by the identification of surface and intracellular proteins, genes, and/or other markers indicative of the lineage commitment of the ASCs to a particular terminal differentiated state. These methods will include, but are not limited to (a) detection of cell surface proteins by immunofluorescent assays such as flow cytometry or in situ immunostaining of ASC surface proteins such as fatty acid binding protein, adipocyte (3101), HSP20-like protein, stathmin, elfin/PDZ, Lim domain protein 1 or leptin as exemplified herein, as well as alkaline phosphatase, CD44, CD 146, integrin beta 1 or osteopontin (Gronthos et al., 1994, Blood 84:4164-4173); (b) detection of intracellular proteins by immunofluorescent methods such as flow cytometry or in situ immunostaining of adipose tissue-derived stromal cells using specific monoclonal antibodies directed against peroxisome proliferator activated receptors, retinoid X receptors, vitamin D receptors or Cbfal; (c) detection of the expression of lineage selective mRNAs such as osteocalcin, PPAR gamma, leptin, Cbfal, interleukin 7, osteoprotegerin ligand and/or macrophage colony stimulating factor, leukocyte marker and growth factor by methods such as polymerase chain reaction, in situ hybridization, and/or other blot analysis (See Gimble et al., 1989, Blood 74:303-311).

Genetically modified ASCs are also useful in the instant invention. Genetic modification may, for instance, result in the expression of exogenous genes (“transgenes”) or in a change of expression of an endogenous gene. Such genetic modification may have therapeutic benefit. In one embodiment, an ASC is genetically modified to express GALC in order to treat Krabbe disease. The GALC used to genetically modify the ASC may be from a human, monkey, mouse or rat. Alternatively, the genetic modification may provide a means to track or identify the modified cells, for instance, after implantation of a composition of the invention into an individual. Tracking a cell may include tracking migration, assimilation and survival of a transplanted genetically-modified cell. Genetic modification may also include at least a second gene. A second gene may encode, for instance, a selectable antibiotic-resistance gene or another selectable marker. Proteins useful for tracking a cell include, but are not limited to, green fluorescent protein (GFP), any of the other fluorescent proteins (e.g., enhanced green, cyan, yellow, blue and red fluorescent proteins; Clontech, Palo Alto, Calif.), or other tag proteins (e.g., LacZ, FLAG-tag, Myc, His₆, and the like). Bromodeoxyuridine is also useful for tracking cells.

The ASCs may be genetically modified using any method known to the skilled artisan. See, for instance, Sambrook et al. (2001, Molecular Cloning: A Laboratory Manual, Cold Spring Harbor Laboratory Press, Cold Spring Harbor, N.Y.), and in Ausubel et al., Eds, (1997, Current Protocols in Molecular Biology, John Wiley & Sons, New York, N.Y.). For example, an ASC may be exposed to an expression vector comprising a nucleic acid including a transgene, such that the nucleic acid is introduced into the cell under conditions appropriate for the transgene to be expressed within the cell. The transgene generally is an expression cassette, including a polynucleotide operably linked to a suitable promoter. The polynucleotide can encode a protein, or it can encode biologically active RNA (e.g., antisense RNA or a ribozyme). Thus, for example, the polynucleotide can encode a gene conferring resistance to a toxin, a hormone (such as peptide growth hormones, hormone releasing factors, sex hormones, adrenocorticotrophic hormones, cytokines (e.g., interfering, interleukins, lymphokines), etc.), a cell-surface-bound intracellular signaling moiety (e.g., cell adhesion molecules, hormone receptors, etc.), a factor promoting a given lineage of differentiation (e.g., bone morphogenic protein (BMP)), etc.

Within the expression cassette, the coding polynucleotide is operably linked to a suitable promoter. Examples of suitable promoters include prokaryotic promoters and viral promoters (e.g., retroviral ITRs, LTRs, immediate early viral promoters (IEp), such as herpesvirus IEp (e.g., ICP4-IEp and ICP0-IEEp), cytomegalovirus (CMV) IEp, and other viral promoters, such as Rous Sarcoma Virus (RSV) promoters, and Murine Leukemia Virus (MLV) promoters). Other suitable promoters are eukaryotic promoters, such as enhancers (e.g., the rabbit (β-globin regulatory elements), constitutively active promoters (e.g., the .beta.-actin promoter, etc.), signal specific promoters (e.g., inducible promoters such as a promoter responsive to RU486, etc.), and tissue-specific promoters. It is well within the skill of the art to select a promoter suitable for driving gene expression in a predefined cellular context. The expression cassette can include more than one coding polynucleotide, and it can include other elements (e.g., polyadenylation sequences, sequences encoding a membrane-insertion signal or a secretion leader, ribosome entry sequences, transcriptional regulatory elements (e.g., enhancers, silencers, etc.), and the like), as desired.

The expression cassette containing the transgene should be incorporated into a genetic vector suitable for delivering the transgene to the cells. Depending on the desired end application, any such vector can be so employed to genetically modify the cells (e.g., plasmids, naked DNA, viruses such as adenovirus, adeno-associated virus, herpesviruses, lentiviruses, papillomaviruses, retroviruses, etc.). Any method of constructing the desired expression cassette within such vectors can be employed, many of which are well known in the art (e.g., direct cloning, homologous recombination, etc.). The choice of vector will largely determine the method used to introduce the vector into the cells (e.g., by protoplast fusion, calcium-phosphate precipitation, gene gun, electroporation, DEAE dextran or lipid carrier mediated transfection, infection with viral vectors, etc.), which are generally known in the art.

II. Therapeutic Methods

In one aspect, the invention provides a method of treating at least one symptom of a leukodystrophy in a mammal, said method comprising administering to said mammal an isolated ASC exhibiting a non-immunogenic characteristic. In one embodiment, the leukodystrophy is Krabbe disease, adrenoleukodystrophy/adrenomyeloneuropathy, Aicardi-Goutieres syndrome, Alexanders disease, childhood ataxia with diffuse central nervous system hypomyelination (CACH), cerebral autosomal dominant arteriopathy with subcortical infarcts and leukoencephalopathy (CADASIL), Canavan disease, cerebrotendinous xanthomatosis, metachromatic leukodystrophy, neonatal adrenoleukodystrophy, ovarioleukodystrophy syndrome, Pelizaeus-Merzbacher disease, Refsum disease, Van der Knaap syndrome or Zellweger syndrome. Preferably, the leukodystrophy is Krabbe disease.

In another aspect, the invention provides a method for increasing the level of GALC in a tissue or a mammal by administering an isolated ASC exhibiting a non-immunogenic characteristic to the mammal. Administration of the ASCs of the invention can occur at various time points. For example, the cells can be administered at the onset of symptoms of the leukodystrophy. In one embodiment, the cells are administered about 1 day, preferably 2 days, more preferably 3 days, preferably 4 days, and more preferably 7 days from the onset of the symptoms. In another embodiment, the cells can be administered to a mammal weeks after the onset of symptoms.

The cells may be administered into a host in a wide variety of ways. Preferred modes of administration are parenteral, intraperitoneal, intravenous, intradermal, epidural, intraspinal, intrasternal, intra-articular, intra-synovial, intrathecal, intra-arterial, intracardiac, intramuscular, subcutaneous, topical, percutaneous, surgical implant, internal surgical paint or infusion pump. In one embodiment, the agent and carrier are administered in a slow release formulation such as a direct tissue injection or bolus, implant, microparticle, microsphere, nanoparticle or nanosphere. Exemplified herein are methods for intravenously administering differentiated ASCs by tail vein injection, but the present invention is not limited to such methods.

The presence of the differentiated cells of the invention may be detected in a subject by a variety of techniques including, but not limited to, flow cytometric, immunohistochemical, in situ hybridization, and/or other histologic or cellular biologic techniques. See, for example, Kopen et al., 1999, Proc Natl Acad Sci 96:10711-10716.

Transplantation of the cells of the present invention can be accomplished using techniques well known in the art as well as those described herein or as developed in the future. The present invention comprises a method for transplanting, grafting, infusing, or otherwise introducing the cells into a mammal. Also, methods for bone transplants are well known in the art and are described in, for example, U.S. Pat. No. 4,678,470, pancreas cell transplants are described in U.S. Pat. No. 6,342,479, and U.S. Pat. No. 5,571,083, teaches methods for transplanting cells to any anatomical location in the body.

In order to transplant the cells of the present invention into a human, the cells are prepared as described herein. In one embodiment, the cells are from the patient for which the cells are being transplanted into (autologous transplantation). In another embodiment, the cells are from a non-human primate, for example a rhesus monkey. One preferable mode of administration is as follows. In the case where cells are not from the patient (allogeneic transplantation), at a minimum, blood type or haplotype compatibility should be determined between the donor cell and the patient. Surgery is performed using a Brown-Roberts-Wells computed tomographic (CT) stereotaxic guide. The patient is given local anesthesia in the scalp area and intravenously administered midazolam. The patient undergoes CT scanning to establish the coordinates of the region to receive the transplant. The injection cannula usually consists of a 17-gauge stainless steel outer cannula with a 19-gauge inner stylet. This is inserted into the brain to the correct coordinates, then removed and replaced with a 19-gauge infusion cannula that has been preloaded with about 30 μl of tissue suspension. The cells are slowly infused at a rate of about 3 μl/min as the cannula is withdrawn. Multiple stereotactic needle passes are made throughout the area of interest, approximately 4 mm apart. The patient is examined by CT scan postoperatively for hemorrhage or edema. Neurological evaluations are performed at various post-operative intervals, as well as PET scans to determine metabolic activity of the implanted cells.

Between about 10⁵ and about 10¹³ cells per 100 kg person are administered to a human. In some embodiments, from 1.5×10⁶ to 1.5×10¹² cells are administered per 100 kg person. In some embodiments, between from 1×10⁹ to 5×10¹¹ cells are administered per 100 kg person. In some embodiments, from 4×10⁹ to 2×10¹¹ cells are administered per 100 kg person. In other embodiments, from 5×10⁸ cells to 1×10¹⁰ cells are administered per 100 kg person. The cells can be administered to a person by various methods including but not limited to infusion and intravenous administration.

In some embodiments, a single administration of the cells is provided. In some embodiments, multiple administrations are provided. In some embodiments, multiple administrations are provided over the course of 3-7 consecutive days. In some embodiments, 3-7 administrations are provided over the course of 3-7 consecutive days. In other embodiments, 5 administrations are provided over the course of 5 consecutive days.

In some embodiments, a single administration of between about 10⁵ and about 10¹³ cells per 100 kg person is provided. In some embodiments, a single administration of between about 1.5×10⁸ and about 1.5×10¹² cells per 100 kg person is provided. In some embodiments, a single administration of between about 1×10⁹ and about 5×10¹¹ cells per 100 kg person is provided. In some embodiments, a single administration of about 5×10¹⁰ cells per 100 kg person is provided. In some embodiments, a single administration of 1×10¹⁰ cells per 100 kg person is provided.

In some embodiments, multiple administrations from 10⁵ to 10¹³ cells per 100 kg person are provided. In some embodiments, multiple administrations from 1.5×10⁸ to 1.5×10¹² cells per 100 kg person are provided. In some embodiments, multiple administrations from 1×10⁹ to 5×10¹¹ cells per 100 kg person are provided over the course of 3-7 consecutive days. In some embodiments, multiple administrations from 4×10⁹ cells per 100 kg person are provided over the course of 3-7 consecutive days. In some embodiments, multiple administrations of 2×10¹¹ cells per 100 kg person are provided over the course of 3-7 consecutive days. In some embodiments, 5 administrations of 3.5×10⁹ cells are provided over the course of 5 consecutive days. In some embodiments, 5 administrations of 4×10⁹ cells are provided over the course of 5 consecutive days. In some embodiments, 5 administrations of 1.3×10¹¹ cells are provided over the course of 5 consecutive days. In some embodiments, 5 administrations of 2×10¹¹ cells are provided over the course of 5 consecutive days.

In one embodiment of the invention, the cells of the present invention are administered to a mammal suffering from a disease, disorder or condition, for example Krabbe disease, involving cells expressing GALC in order to augment or replace the diseased or damaged cells. ASCs are preferably administered to a human suffering from a disease, disorder or condition characterized as a leukodystrophy. The precise site of administration of the cells depends on any number of factors, including but not limited to, the damaged area to be treated, the type of disease being treated, the age of the human and the severity of the disease, and the like. Determination of the site of administration is well within the skill of the artisan versed in the administration of such cells. Based on the present disclosure, the cells can be administered to the patient via intravenous routes.

There are several ways in which ASCs can be used in a mammal, preferably a human, to treat leukodystrophies. For example, the cells can be used as precursor cells that differentiate following introduction into the patient or as cells which have been differentiated into leukocytes or fibroblasts, for example, prior to introduction into the patient. In either situation, the cells can be differentiated to express at least one protein characteristic of a leukocyte or fibroblast, for example, including, but not limited GALC. In one embodiment, the ASC optionally differentiates in vivo into a cell that expresses GALC. In another embodiment, the ASC is cultured in vivo for a period of time without being induced to differentiate prior to the administration of the ASC to the mammal.

The present invention now will be described more fully by the following examples. This invention may, however, be embodied in many different forms and should not be construed as limited to the embodiments set forth herein; rather, these embodiments are provided so that this disclosure will be thorough and complete, and will fully convey the scope of the invention to those skilled in the art.

### EXAMPLES

### Example 1

### Adipose Stromal Cell (ASC) Isolation and Differentiation

In order to determine whether ASCs underwent adipogenesis, a culture system was established using adult stromal cells cultured in the presence of dexamethasone, insulin, isobutylmethylxanthine and a thiazolidinedione followed by culture in the presence of dexamethasone and insulin.

The materials and methods used in the experiments presented in this Example are now described.

Isolation of Adipose Stem Cells: It has been demonstrated that human adipose tissue is a rich source of stromal-like adult stem cells (Gimble et al., 2003, Curr. Top Dev. Biol. 58:137-160; Aust et al., 2004, Cytotherapy 6(1):7-14; Awad et al., 2003, Tissue Eng. 9(6):1301-1312; Awad et al., 2004, Biomaterials 25(16):3211-3222; Elmslie et al., 2000, J. Clin. Psychiatry 61(3):179-184; Delany et al., 2005, Mol. Cell. Proteomics 4:731-740; Gronthos et al., 2001, J. Cell Physiol. 189(1):54-63; Halvorsen et al., 2000, Int. J. Obes. Relat. Metab. Disord. 24 Suppl. 4:S41-44; Halvorsen et al., 2001, Metabolism 50(4):407-413; Halvorsen et al., 2001, Tissue Eng. 7(6):729-741; Hicok et al., 2004, Tissue Eng. 10(3-4):371-380; Safford et al., 2002, Biochem. Biophys. Res. Commun. 294(2):371-379; Safford et al., 2004, Exp. Neurol. 187(2):319-328; Sen et al., 2001, J. Cell Biochem. 81(2):312-319; Wickham et al., 2003, Clin. Orthop. (412):196-212; Guilak et al., 2005, J. Cell Physiol.; Mitchell et al., Stem Cells online Jan. 12, 2006:2005-0235.)

Based on the original methods described by Hauner and others (Hauner et al., 1989, J. Clin. Invest. 84(5):1663-1670; Hauner et al., 1988, Horm. Metab. Res. Suppl. 19:35-39), reproducible and efficient methods have been developed to isolate adult stem cells from human liposuction tissue (Aust et al., 2004, Cytotherapy 6(1):7-14; Halvorsen et al., 2001, Metabolism 50(4):407-413; Mitchell et al., Stem Cells online Jan. 12, 2006:2005-0235). The procedure involved collagenase digestion of the tissue, differential centrifugation, and expansion in culture. In an analysis of specimens obtained from 42 individual donors, a mean of 247,401±136,514 human adipose-derived stem cells (huASCs) was recovered from a single ml of liposuction waste within a 6.0±2.4 day expansion period. The demographic profile of the tissue donor population (n=120 patients) is summarized in Table 1.

TABLE 1
Donor Demographics

| | BMI < 25 | 25 < BMI < 30 | 30 < BMI < 35 | BMI > 35 |
|---|---|---|---|---|
| Mean BMI ± S.D. | 22.3 ± 1.4 | 27.5 ± 1.4 | 32.3 ± 1.3 | 36.9 ± 1.3 |
| BMI Range | 19.9-24.7 | 25.1-29.9 | 30.1-34.2 | 35.3-39.2 |
| Total # of Subjects | 57 | 73 | 13 | 7 |
| # Caucasian | 51 | 34 | 11 | 5 |
| # African/Am | 5 | 2 | 0 | 0 |
| # Asian | 1 | 5 | 2 | 2 |
| # Hispanic | 0 | 2 | 0 | 0 |
| Female | 98%(56) | 86%(37) | 85%(11) | 71%(5) |
| Male | 2%(1) | 14%(6) | 15%(2) | 29%(2) |
| Mean Age | 38.9 ± 8.4 | 41.1 ± 11.4 | 42.7 ± 14.8 | 34.1 ± 10.7 |
| Age Range | 24-62 | 18-64 | 25-63 | 22-50 |

After passage in vitro, these cells, which were identified as ASCs, exhibit a differentiation potential comparable to that of bone marrow derived Mesenchymal Stem Cells (MSCs) (Table 2) (Aust et al., 2004, Cytotherapy 6(1):7-14; Halvorsen et al., 2001, Metabolism 50(4):407-413; Mitchell et al., Stem Cells online Jan. 12, 2006:2005-0235). Other groups have reported similar findings 78-85. Flow cytometric analysis has been used as an initial proteomic approach to define the SC's immunophenotype.

TABLE 2
Characterization of huASCs (Passage 2)

| | Surface | Differen- |
|---|---|---|
| | Negative | tiation |
| Surface Positive Markers | Markers | Potential |
| CD9, CD10, CD13, CD29, CD44, | CD11, CD14, | Adipocyte |
| CD49d, CD54, CD55, CD59, CD71, | CD16, CD18, | Chondrocyte |
| CD73, CD90, CD105, CD106, | CD31, CD45, | Hematopoietic |
| CD146, CD166, α-smooth muscle | CD50, CD56, | Support |
| actin, collagen type I, collagen | CD62, CD104, | Myocyte |
| type III, HLA-ABC, nestin, | Factor VIII | (Cardiac, |
| osteopontin, osteonectin, | related | Skeletal) |
| vimentin | Ag, HLA-DR | Myofibroblast |
| | | Neuronal |
| | | Osteoblast |

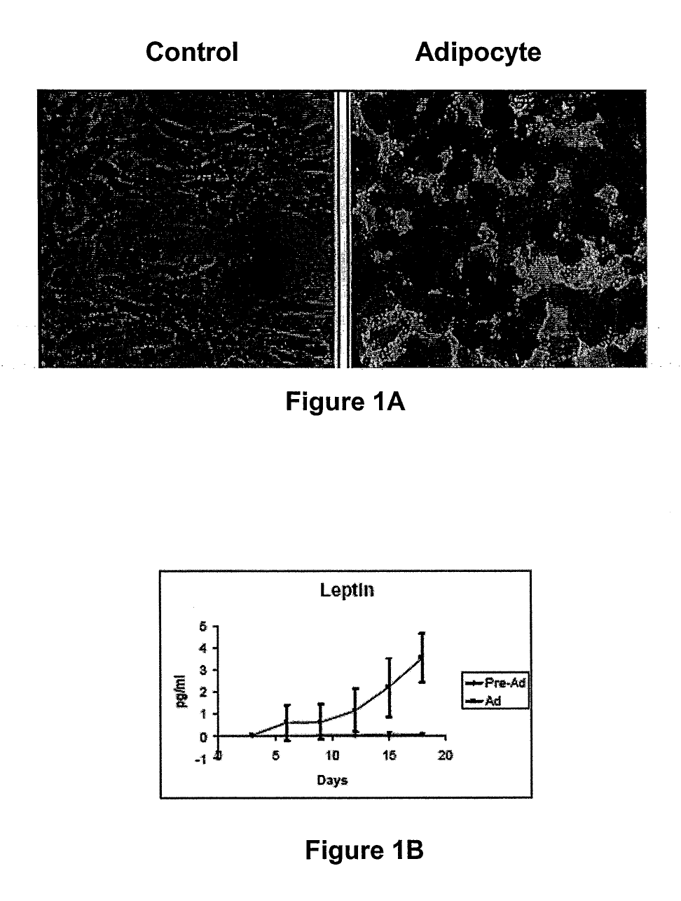

Adipogenesis: Confluent stromal cell cultures were induced for 3 days with dexamethasone, insulin, isobutylmethylxanthine and a thiazolidinedione followed by culture in the presence of dexamethasone and insulin. After 14 days in culture, the cells were fixed and stained for neutral lipid with Oil Red O (FIG. 1, left panel) and the conditioned medium assayed for leptin levels by ELISA days (FIG. 1, right panel).

The results of the experiments presented in this Example are now described.

In the presence of dexamethasone, insulin, isobutylmethylxanthine and a thiazolidinedione, the huASCs underwent adipogenesis (FIG. 1). The cells accumulated lipid vacuoles, which were stained for neutral lipid with Oil Red O dye (FIG. 1A), and expressed adipocyte-specific markers, including the secreted cytokine leptin (FIG. 1B) and the fatty acid binding protein aP2 (Halvorsen et al., 2001, Metabolism 50(4):407-413; Sen et al., 2001, J. Cell Biochem. 81(2):312-319). Moreover, the cells displayed a lipolytic response to adrenergic compounds, a biochemical characteristic of mature primary adipocytes (Halvorsen et al., 2001, Metabolism 50(4):407-413).

These results show that adult stromal cells are capable of undergoing adipogenesis.

### Example 2

### Multipotentiality of ASCs

In order to determine if non-human primate ASCs are capable of multipotential differentiation, non-human primate ASCs were cultured in the same conditions as described for huASCs and then studied based upon morphology, proliferation potential and differentiation potential.

The materials and methods used in the experiments presented in this Example are now described.

Multipotentiality of huASCs: The differentiation potential of the huASCs was not limited to the adipocyte lineage. Conditions were developed that promote huASCs differentiation along the chondrocyte and osteoblast pathways (Awad et al., 2003, Tissue Eng. 9(6):1301-1312; Awad et al., 2004, Biomaterials 25(16):3211-3222; Wickham et al., 2003, Clin. Orthop. (412):196-212; Guilak et al., 2005; J. Cell Physiol., Erickson et al., 2002, Biochem. Biophys. Res. Commun. 290(2):763-769; Wang et al., 2005, J. Cell Physiol.). When suspended in calcium alginate and incubated in the presence of ascorbate, dexamethasone, and transforming growth factor α, huASCs exhibited an induction in chondrogenic markers, including collagen types II and VI and proteoglycans (Awad et al., 2003, Tissue Eng. 9(6):1301-1312; Awad et al., 2004, Biomaterials 25(16):3211-3222; Wickham et al., 2003, Clin. Orthop. (412):196-212; Erickson et al., 2002, Biochem. Biophys. Res. Commun. 290(2):763-769). When cultured in the presence of 1,25 dihydroxyvitamin D3, dexamethasone, ascorbate, and α-glycerophosphate, huASCs secreted osteocalcin and mineralized their extracellular matrix, hallmarks characteristic of osteoblast function (Halvorsen et al., 2001, Tissue Eng. 7(6):729-741; Hicok et al., 2004, Tissue Eng. 10(3-4):371-380; Guilak et al., 2005, J. Cell Physiol.). In vivo, huASCs combined with a hydroxyapatite biomaterial synthesize osteoid matrix when implanted subcutaneously into immunodeficient mice (Hicok et al., 2004, Tissue Eng. 10(3-4):371-380; Justesen et al., 2004, Tissue Eng. 10(3-4):381-391).

There is substantial data that demonstrates that huASCs and murine ASCs cultured in the presence of antioxidants undergo morphologic and phenotypic changes consistent with neuronal differentiation (Safford et al., 2002, Biochem. Biophys. Res. Commun. 294(2):371-379; Safford et al., 2004, Exp. Neurol. 187(2):319-328; Ashjian et al., 2003, Plast. Reconstr. Surg. 111(6):1922-1931). The list of neuronal markers expressed by huASCs has been extended to include nestin, GFAP, S-100, NeuN, MAP2, GABA, the NR-1 and 2 subunits of the glutamate receptor, as well as voltage gated calcium channels (Safford et al., 2004, Exp. Neurol. 187(2):319-328). It has also been found that huASCs also secrete a number of cytokines and support hematopoiesis in vitro (R W Storms, J M Gimble, M S in preparation). Furthermore, it has been documented that huASC clones retain their multipotentiality.

The results of the experiments presented in this Example are now described.

It was found that the stromal vascular fraction of adipose tissue contained a high frequency of lineage specific colony forming units (CFU) (Mitchell et al., Stem Cells online Jan. 12, 2006:2005-0235). Mean values from n=7 to 12 donors were as follows: CFU-F (fibroblast), 1 per 30 cells; CFU-ALP (alkaline phosphatase), 1 per 285 cells; CFU-Ad (adipocyte), 1 per 40 cells; and; CFU-Ob (osteoblast), 1 per 12 cells. With progressive passage, the frequency of the individual lineages was enriched by approximately 10-fold. These values exceed those estimated for bone marrow-derived MSCs by 2-3 orders of magnitude.

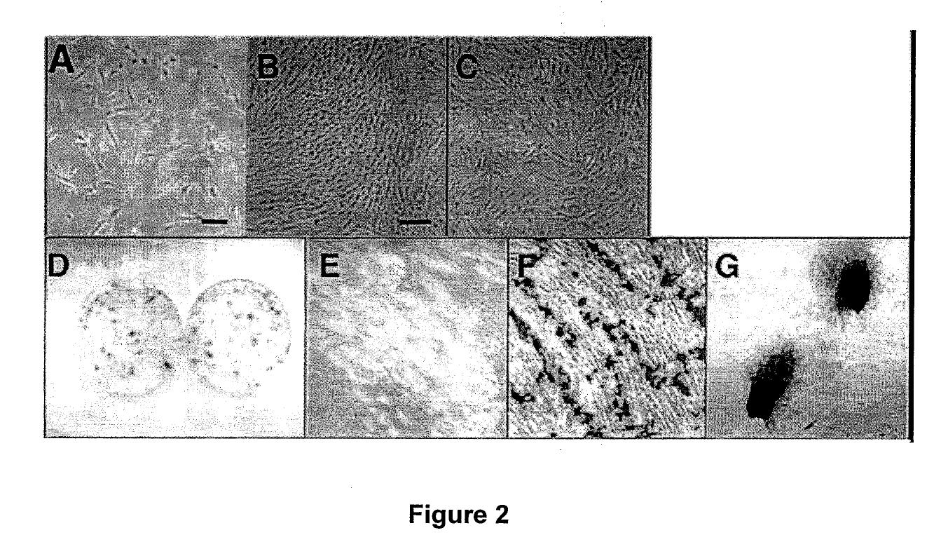

FIG. 2 illustrates the morphology, proliferation and differentiation potential of non-human primate ASCs (pASCs). Culture expanded non-human primate ATSCs exhibited typical spindle-shaped fibroblastic morphology (FIG. 2A for low density and FIG. 2B for high density). Compared to pATSCs, primate bone marrow stem cells (pBMSCs) were more heterogeneous and had a fibroblastic morphology (C). A single cell was expanded into a clonal population and generated colony forming units (CFUs) that were evident following Giemsa staining (FIG. 2D). pATSCs that were passaged 3-4 times retained multilineage differentiation capability undergoing adipogenesis (FIG. 2E), osteogenesis (FIG. 2F) and chondrogenesis (FIG. 2G). The ASCs isolated from subcutaneous adipose tissue of non-human primates (prASCs) were multipotent in a manner similar to the huASCs. Clonal passages of the prASCs differentiated along the adipocyte, chondrocyte, osteoblast, and neuronal pathways (FIG. 2).

These results suggest that ASCs from non-human primate and other large animal models can serve as human surrogates in pre-clinical tests.

### Example 3

### Proteomic Analysis of Adipose Stem Cells

In order to determine the expression profile of differentiated huASCs as compared to undifferentiated huASCs, proteomic analysis was performed on these cells.

The materials and methods used in the experiments presented in this Example are now described.

Dimensional-Polyacrylamide Gel Electrophoresis: Protein analyses relied upon traditional 2D-electrophoresis to initially separate complex mixtures of proteins. Samples were solubilized in a solution comprising 8M urea, 4% CHAPS, 65 mM DTT, 40 mM Tris. Following centrifugation to remove unsolubilized material, 333-500 μg of protein was mixed with rehydration buffer (8M urea, 4% CHAPS, 1% IPG buffer, 0.3% DTT) and introduced into the dry IPG strips (typically 18 cm, pH 4-10NL) under conditions of active rehydration (e.g. with a slight voltage applied across the strips). Proteins were focused at a maximum 10,000 V for a total of 90,000 v-h. Upon completion of 1st dimension electrophoresis, the IPG strips were either directly subjected to 2nd dimension SDS-PAGE or frozen at −80° C. for later analysis. For the 2nd dimension, the IPG strips were equilibrated first with 50 mM Tris-HCL, pH 8.8, 6M urea, 30% glycerol, 2% SDS, 1% DTT for 15 minutes followed by a second equilibration with 50 mM Tris-HCL, pH 8.8, 6M urea, 30% glycerol, 2% SDS, 5% iodoacetamide for 15 minutes. The strips were rinsed with electrophoresis buffer (25 mM Tris, 190 mM glycine, 0.1% SDS) and then embedded in low-melting temperature agarose onto the top of 25×20 cm 12% acrylamide gel. Gels were run at constant voltage until the bromophenol blue dye front reached the bottom of the gel.

Protein Staining and Quantitation: Following 2D-electrophoresis, gels were stained with Sypro Ruby. The stained gels were scanned with a Molecular Imager FX with data directly imported into PDQuest. For each gel, the relative abundance of each resolved protein feature was quantified by mathematical fitting of Gaussian curves in two dimensions. Data within each were normalized (either expressed as a percentage of total spot abundance, or relative to a set of housekeeping proteins) and routine statistical analyses were available within the software package (identification of unique spots, absent spots, or spots up or down regulated under specified conditions). However, data were typically exported in Excel spreadsheet format for statistical analyses.

Mass Spectroscopy Protein Identification: All proteomic studies were conducted in accordance with the guidelines proposed by the editors of Molecular & Cellular Proteomics 101 and proteomics techniques well known in the art, as described, for example, in the following textbooks, the contents of which are hereby incorporated by reference: Proteome Research: New Frontiers in Functional Genomics (Principles and Practice), M. R. Wilkins et al., eds., Springer Verlag, 1007; 2-D Proteome Analysis Protocols, Andrew L Link, editor, Humana Press, 1999; Proteome Research: Two-Dimensional Gel Electrophoresis and Identification Methods (Principles and Practice), T. Rabilloud editor, Springer Verlag, 2000; Proteome Research: Mass Spectrometry (Principles and Practice), P. James editor, Springer Verlag, 2001; Introduction to Proteomics, D. C. Liebler editor, Humana Press, 2002; Proteomics in Practice: A Laboratory Manual of Proteome Analysis, R. Westermeier et al., eds., John Wiley & Sons, 2002.

Following electrophoresis, staining, scanning, spot detection, and match set preparation, proteins of interest were selected and their standard spot numbers were entered into a “Cut List.” This “Cut List” was used by the spot cutter (under control of PDQuest 7.2.0) to automatically select and excise the protein features in order of least to most abundant from one or more gels. Excised gel plugs were deposited into a 96 well plate with sample tracking maintained by PDQuest 7.2.0 in conjunction with ProteinLynx Global Server. The plate was transferred to the MassPrep station where the proteins within the gel plugs were automatically destained, reduced, alkylated, dehydrated, rehydrated and digested with trypsin. The resulting peptides were extracted, cleaned-up, and then deposited onto MALDI plates and into 96 well plates (for the Q-TOF). Peptide mass fingerprints were determined by matrix assisted laser desorption—time of flight (MALDITOF) mass spectroscopy. The generated peptide mass fingerprints were used to interrogate the SwissProt, TREMBL, or NCBI databases to tentatively identify known proteins. If a spot could not be identified by MALDITOF or if there was some ambiguity in protein assignment, the peptides were separated by capillary liquid chromatography interfaced to an ESI-MS/MS MicroMass Q-TOF mass spectrometer. The derived partial de novo sequences from the peptides were used to interrogate protein, genome, or EST databases for unequivocal protein identification. Whenever multiple gel features were found to identify the same protein, under one or multiple names, the protein was entered into the database only as a single entity. PDQuest 7.2.0 software in conjunction with the WorksBase data management system tracked the processing of all samples and individual spots from the initial identification through statistical analyses, spot excision, preparation for mass spectroscopy, and protein identification. Data from the mass spectroscopy analysis were back annotated to the original gel image such that clicking on the spot of interest revealed its identity, peptide mass spectra, derived amino acid sequence, and pre-selected data downloaded from public databases. To facilitate the analysis of the large amounts of data produced in these experiments, GenMAPP (Gene MicroArray Pathway Profiler) & MappFinder (genmapp.org/) was used to examine the data. GenMAPP is a computer application designed to visualize gene expression data on maps representing biological pathways and groupings of genes.

The results of the experiments presented in this Example are now described.

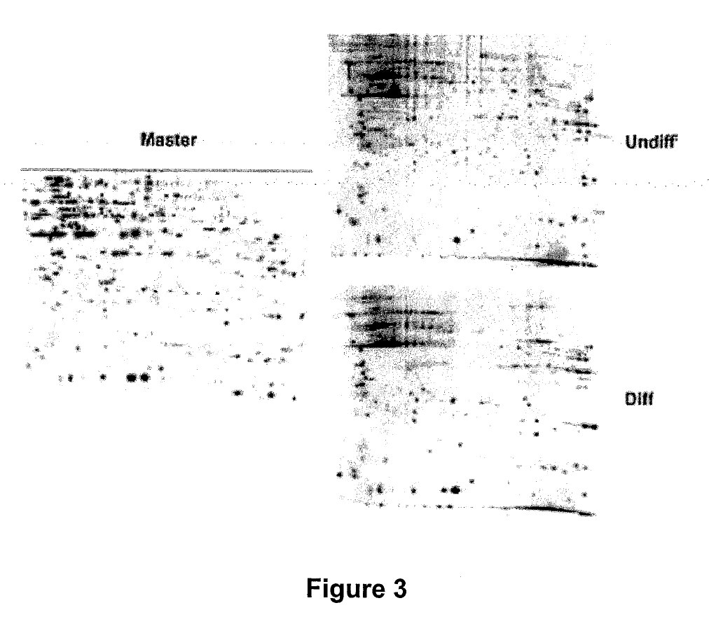

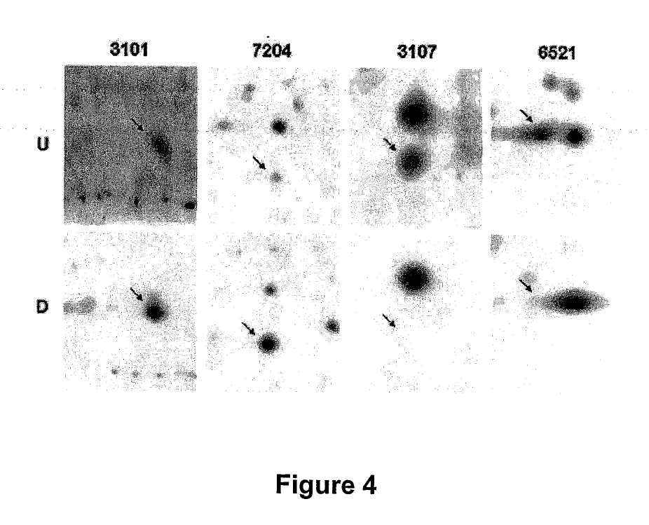

The proteome of huASCs in the undifferentiated and adipocyte differentiated states was compared using 2-dimensional polyacrylamide gel electrophoresis (2D-PAGE) and tandem mass spectroscopy (FIG. 3) (Delany et al., 2005, Mol. Cell Proteomics 4:731-740). More than 430 Sypro stained spots on 2DPAGE gels was distinguished from both undifferentiated and adipocyte huASCs (of which 288 were shared in common) and over 170 individual proteins were identified and expressed by the undifferentiated huASCs by mass spectroscopy (Table 1) (Delany et al., 2005, Mol. Cell Proteomics 4:731-740). Following adipogenesis, the levels of over 40 proteins were upregulated by >2-fold while an additional 13 proteins were reduced by >3-fold (FIG. 4 and Table 2) (Delany et al., 2005, Mol. Cell Proteomics 4:731-740).

FIG. 4 illustrates the individual protein features from undifferentiated (U) and adipocyte differentiated (D) huASCs. Differentiation-dependent changes are identified by arrows for fatty acid binding protein, adipocyte (3101), HSP20-like protein (7204), stathmin (3107), and elfin/PDZ and Lim domain protein 1 (6521) on a 2D-PAGE analysis of total huASC lysates.

These results suggest that differentiated huASCs have a different expression profile as compared to undifferentiated huASCs further supporting the multipotential nature of cultured huASCs.

### Example 4

### Characterization of Krabbe Disease Affected-Animals

In order to determine if a rhesus monkey is a suitable model in which to study human Krabbe disease, affected animals were examined thoroughly and the results were compared to the clinical progression of the disease in humans.

The materials and methods used in the experiments presented in this Example are now described.

Growth of Animals: The growth of the developing rhesus monkey (Macaca mulatta) primate fetuses were monitored during pregnancy using monthly ultrasound to assess any growth retardation or changes during pregnancy as well as monitor abortion rate, fetal death, and or problems that might lead to difficult births. In general, animals were allowed to deliver naturally. Once the infants were born, a blood draw was performed for the determination of the genetic status (wild-type, heterozygote or homozygous affected) and a sex check was scheduled and health of all infants as well as the health of the mothers were examined. Genomic DNA was extracted from peripheral blood mononuclear cells and a diagnostic PCR protocol, including a restriction digest, was performed to determine the Krabbe status (heterozygous carrier, homozygous affected or normal). The heterozygous and homozygous infants were kept with their mothers, and the normal animals were released from the project.

Neuroimaging: All affected infants along with age-matched carrier and non-carrier controls were studied monthly using MRI. The MRI sequences included a saggital T1-weighted scan, saggital T2-weighted scan, axial T1-weighted scan, axial proton density, axial T2-weighted scan, coronal T2-weighted scan, and postcontrast axial and coronal T1-weighted scans. When needed, Prohance (Gadoteridol) was administered to the animals at 0.1 mmole/kg intravenously for contrast. The MRI interpretation included the location, extent, and morphology of white matter disease, as well as associated intracranial cortical and ventricular changes.

The data from early magnetic resonance (MR) imaging analysis indicated that no definite anomalies were observed. The affected animals exhibited no abnormal changes in either their grey or white matter, when compared to age matched controls. In addition, the ventricle system and cortical sulci were unremarkable. It is important to note that the animals that were thoroughly analyzed had to be necropsied at approximately 100 days of life. It is plausible that changes in the CNS occur later in life. In support of this theory, one Krabbe animal that lived over 22 months was noted to have increased areas of T2 signal associated with the trigonal area of the posterior horns of the lateral ventricles. In humans, this is often described as an early finding of Krabbe disease.

EMG nerve conductivity studies were performed and infant behavior testing was performed. In addition, these infants were videotaped to document behavior, movement and difficulties these infants have in everyday situations. The animal's weight was also monitored, and their eating habits and general overall health were assessed. Once a significant weight loss was detected and/or severe respiratory difficulties animals were euthanized and a complete necropsy was performed.

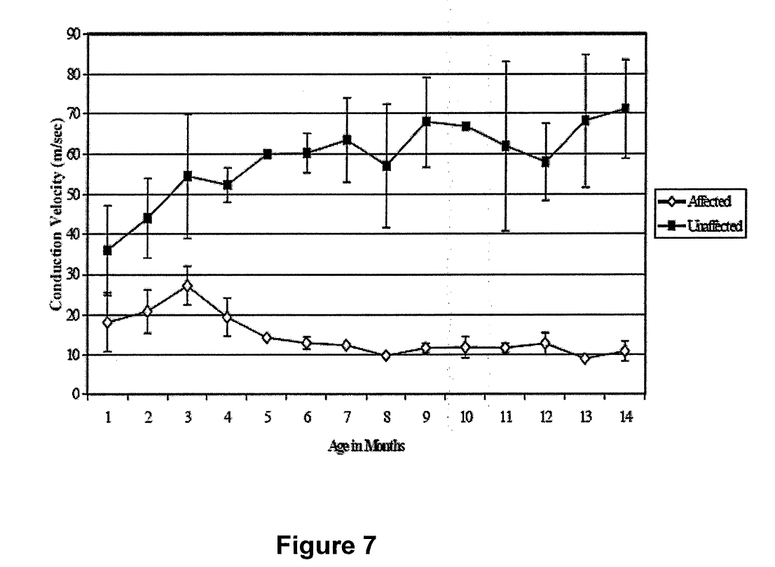

Neurophysiology: Nerve conduction studies were performed as previously described (England et al., 1997, Ann. Neurol. 41(3):375-384). If any affected infants were born, these infants were monitored with magnetic resonance imaging (MRI). Serial nerve conduction studies beginning within the first 2 months of life in 4 homozygous, 2 heterozygous and 2 normal rhesus monkeys to characterize the peripheral neuropathy were performed. Because there were no significant differences between the carrier and normal groups, these groups were combined to create the unaffected comparison group for all subsequent analyses. For each nerve, a significant interaction effect of group by age was found (Median nerve: F(1,57)=24.06, p<0.0001; Tibial nerve: F(1,58)=26.44, p<0.0001; Ulnar nerve: F(1,59=28.68, p<0.0001). Mean conduction velocities of the median, ulnar, and tibial nerves were significantly slower in the affected as compared with the unaffected monkeys at all ages (P<0.0001) (FIG. 7, Weimer et al., 2005, Muscle Nerve 32(2): 185-190). In the affected monkeys, bilateral median, ulnar, and tibial motor nerve conduction studies exhibited normal compound muscle action potential amplitudes, but all nerves exhibited severe prolongation of distal latencies and severe slowing of conduction velocities. F-waves were well formed and reproducible, but latencies were severely prolonged in affected monkeys. The conduction velocity differences became more apparent between the affected and unaffected animals as the monkeys aged. There was no evidence of excessive temporal dispersion in any nerve. In the affected monkeys, the degree of conduction slowing was remarkably uniform along all segments of each nerve and was highly concordant between nerves. When compared to the unaffected monkeys, the serial conduction velocities suggested the occurrence of dysmyelination followed by demyelination in the affected monkeys. These findings are diagnostic of a severe primarily demyelinating polyneuropathy and are in accordance with the expected electrophysiological phenotype of GLD. The diffuse and uniform slowing of motor nerve conduction velocities are typical of an inherited demyelinating (hypomyelinating) neuropathy secondary to a defect in myelination of the peripheral nervous system.

Lipid Analysis: The lipid profile of samples of brain and kidney from two of the affected infants (AA54 and V539) and from three control rhesus monkeys was determined using the method described by Fujita (Fujita et al., 1996, Hum. Mol. Genet. 5(6):711-725).

Samples of both gray and white matter were separated from each other as well as possible before extraction. Psychosine was analyzed on 20-40 mg tissue samples using a high pressure liquid chromatography method with separation on a reverse-phase column and fluorometric detection.

Galactocerebrosidase Activity: Biochemical assays of galactocerebrosidase enzyme activity were performed with the undifferentiated prASCs and huASCs as a function of passage. Levels of enzyme activity was compared to that detected in peripheral blood mononuclear cells obtained from primates and humans.

Behavior Assessment of Infants: The Infant Neurobehavioral Assessment (Early Infancy Assessment; see (Schneider et al., 1991, Am. J. Primatol. 25:137-155), for a description of the tool) was administered to affected animals at 14 and 30 days of age.

A typical protocol for administering Infant Neurobehavioral Assessment Scale (NBAS) and Bayley Scales of Infant Development is as follows:

At least two trained individuals are involved in the testing. One person is responsible for holding the infant and the other person administers the test recording responses to test items. All items necessary for the testing are laid out before the infant is brought to them room and kept in easy reach of the examiner. All testing is done in a quiet, well lit area. Signage is placed on doors stating “Do not disturb, testing going on”.

If an infant is with its mother, the mother is anesthetized with an intramuscular injection of ketamine hydrochloride (10 mg/kg) by a trained animal care or veterinary technician. The infant is removed from the mother, wrapped in a towel, and brought to the examiner.

The examiner keeps the infant wrapped in a towel from the waist down, leaving the arms free to move. Items are administered in the order with orientation/cognitive items done first followed by neuromotor function items and temperament/behavior items. The goal is to maintain the infant in a quiet, alert state throughout the testing period; therefore, the examiner may intervene to console the infant as necessary and appropriate.

The NBAS takes approximately 20 minutes to administer and may be used on infants up to 30 days of age (recommended testing days are 7, 14 and 28). The Bayley Scales of Infant Development requires approximately 10 minutes to administer and may be used between the ages of 2 to 12 months.

Upon completion of testing, the infant is returned to its mother.

The results of the experiments presented in this Example are now described and are summarized in Table 3.

TABLE 3
Summary of clinical outcome of rhesus
monkey Krabbe disease infants

| Animal | | | |
|---|---|---|---|
| # | Life Span | | Clinical Signs of Disease at Necropsy |
| H463 | 76 | days | Initial animal, described postmortem. |
| V539 | 159 | days | Central nervous system (CNS) signs which |
| | | | consisted of severe muscle tremors of head |
| | | | and limbs, ataxia, and hypermetria. |
| AA54 | 12 | days | CNS signs consisting of muscle tremors, |
| | | | especially involving the head, and ataxia. |
| CF36 | Stillborn | | Diagnosed prenatally by chorionic villus |
| | | | sampling |
| C180 | 190 | days | Severe muscle tremors, especially involving |
| | | | the head, and ataxia. Double inspiratory |
| | | | effort which progressively worsened. |
| DG51 | 103 | days | Pronounced muscle tremors, difficulty |
| | | | ambulating, respiratory difficulties |
| DH31 | 21 | months | Severe body tremors, difficulty in ambulat- |
| | | | ing, ataxic and hypermetric and exopthalmia |
| EA75 | 22 | months | Moderate whole body tremors, exaggerated |
| | | | gait, CNS symptoms, facial paralysis |
| EJ72 | 52 | days | Clenched hands and feet, unable to hold up |
| | | | head, no use of legs/arms, no vocalizations |

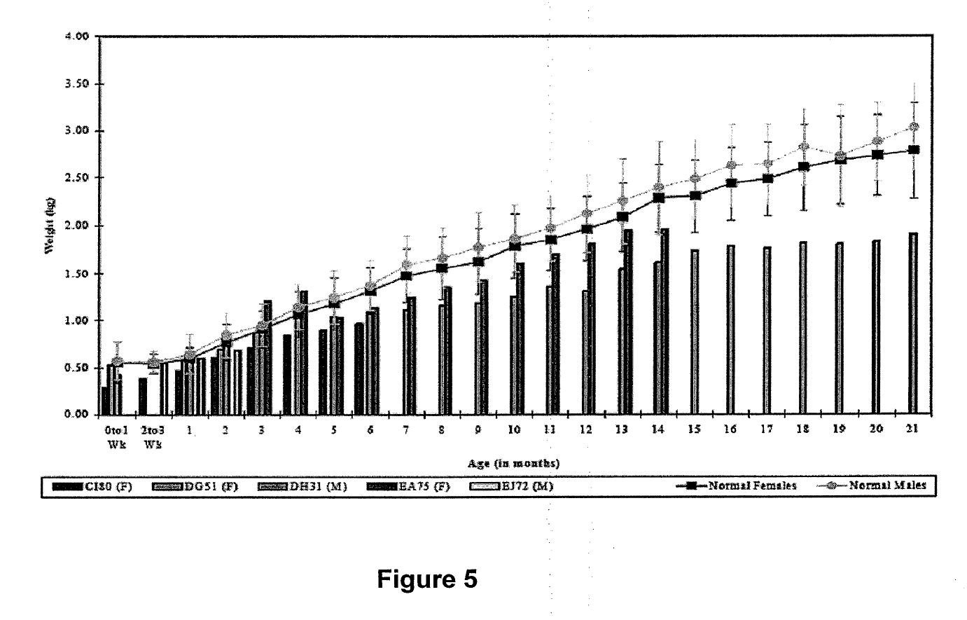

Ultrasound monitoring of fetal animals demonstrated no significant changes in fetal growth or development between normal, carrier and affected animals. However, due to the limited number of affected animals produced to date, it was essential to continue the ultrasound examination of fetal animals for both developmental anomalies and also for the detection of fetal demise. As Krabbe animals were born into the colony, their weight was monitored as part of the routine animal husbandry. FIG. 5 illustrates a comparison of the weights of each affected animal to those of unaffected/normal males and females. In general, affected infants fail to gain weight at the same rate as normal animals of the same age and sex.

Chorionic villus sampling (CVS) can routinely be obtained from the placentas of pregnant monkeys and this type of sampling is an integral part of the natural breeding program. The technology to perform the PCR-based molecular diagnostics routinely for the Krabbe mutation (two base pair deletion) has been developed.

The progression of disease in all of the affected animals was followed with daily Table 4 provides a summary of the clinical observations for infants with Krabbe disease.

TABLE 4
Unique Features of the Krabbe Rhesus Macaque Animal Model

| | |
|---|---|
| 1 | Development of fetal monkey nearly identical to human fetal |
| | development |
| 2 | Development and maturation of CNS homologous to humans |
| 3 | Organization of CNS similar to humans |
| 4 | Timing of myelination during development very similar to humans |
| 5 | Similarity of T1 and T2-weighted MRI images |
| 6 | Results of gene therapy studies indicative of outcomes in humans |
| 7 | Bone marrow transplantation and apheresis procedures can be |
| | performed |
| 8 | Opportunity to investigate efficacy for therapeutic interventions, |
| | plus safety and toxicity |
| 9 | Application of human-based memory, motor function, cognition and |
| | behavioral tests |

Content of galactosylsphingosine (psychosine) in rhesus macaque tissues: Lipid analysis revealed a striking increase in the levels of psychosine in the brain and kidney of both affected infants (Table 5). In cerebral white matter the level of psychosine was raised over 20-fold from normal to about 3,500 pmol/mg protein, while the concentrations of other myelin lipids were reduced. Galactosylceramide concentration was less than normal, but the ratio of galactosylceramide/sulfatide was normal and there was a marked reduction in sphingomyelin with longer-chain fatty acids. On the other hand, the cerebral cortex gray matter exhibited a normal pattern of major lipids and only a small increase in the level of psychosine, which may be due to a small amount of contaminating white matter. In the kidney from the affected monkeys, galactosylceramide concentration was not significantly increased, but on thin layer chromatography, the hydroxy-fatty acid fraction exhibited the presence of a band absent in controls. A relatively large amount of psychosine (0.1 nmol/mg protein) was found, compared to an undetectable level in the control kidney taken from a normal animal.

TABLE 5
Content* of galactosylsphingosine (psychosine)
in rhesus macaque tissues

| | GRAY MATTER | WHITE MATTER |
|---|---|---|
| AA54 (12 days) | 20 | 840 |
| V539 (158 days) | 115 | 3500 |
| Controls | | |
| Normal (newborn) | <2 | |
| Normal (4.5 years) | 25* | 160 |
| Carrier (5.4 years) | 3 | 85 |

*pmole/mg protein;
**May contain some white matter as judged by gal-cer content

GALC Enzyme Activity: Previously the colony had been screened by measuring GALC activity in leukocytes. While the leukocytes of some of the monkeys contained less GALC activity than others, it was not possible to conclusively identify carriers by enzyme analysis. GALC activity was measured in the 2 homozygous affected, 21 normal and 20 carrier monkeys as previously described (Wenger et al., 1991, New York: Wiley-Liss). The 2 affected infants had a GALC activity less than 2% of normal in leukocytes and cultured skin fibroblasts. The average GALC activity for 21 PCR-confirmed non-carrier rhesus monkeys was 0.94 nmol/h/mg of protein, while the average for 20 PCR-identified carriers was 0.52 nmol/h/mg of protein. As would be predicted, the average amount of GALC activity from the carrier animals was approximately one-half of the average amount of GALC activity from the wild-type animals, since carrier animals possess only one functional gene. Similar to the situation in humans, a wide range of values for GALC activity in both normal and carrier rhesus monkeys was observed. The range of values for GALC activity for normal individuals was 0.39 to 1.6 nmol/h/mg of protein, while the range for carriers was 0.2 to 1.1 nmol/h/mg of protein. In fact, thirteen carrier monkeys exhibited a GALC activity that was higher than that of the lowest value in a normal animal. This has made unequivocal identification of carrier monkeys by biochemistry alone problematic and illustrates the value of mutation-based carrier testing in such an inbred population.

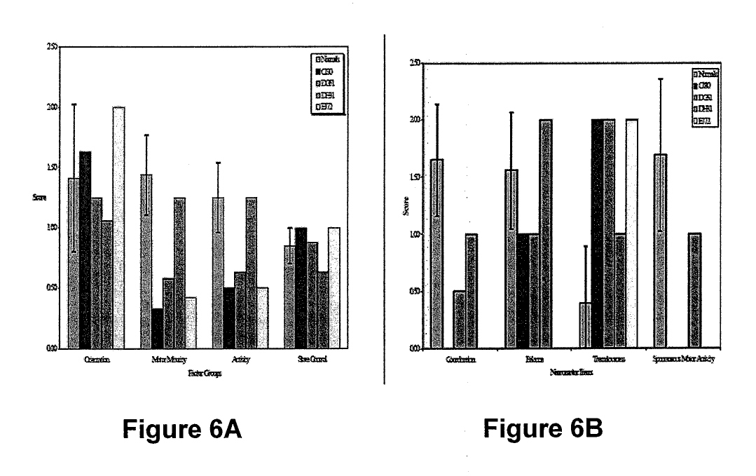

Behavior Assessment of Infants: The Infant Neurobehavioral Assessment (Early Infancy Assessment; see Schneider et al., 1991, Am. J. Primatol. 25:137-155 for a description) was administered to affected animals at 14 and 30 days of age. Since two animals in the colony were not identified as being affected until later, these affected infants (DG51 and DH31) were only tested at 30 days of age. As controls, 20 normal infants who were being mother-reared were also tested at each time point. The composite scores for testing clusters at 30 days are presented in FIG. 6. At all time points, composite scores on the motor cluster and activity cluster were considerably lower (greater than one standard deviation below) for animals CI80, DG5, and EJ72 when compared with the normal monkeys. In terms of orientation and state control, all of the animals were within the normal ranges at all time points tested.

Individual test items that measure neuromotor maturity were also examined (FIG. 6, right panel). The affected infants exhibited poorer coordination, lacked the ability to maintain balance, failed to demonstrate normal levels of spontaneous motor activity, had barely discernable resistance to passive flexion and extension of limbs, and could not withstand moderate resistance i.e., a decreased strength of muscles when actively contracting. The Krabbe infants also exhibited a significant amount of tremulousness. Based on items that measure temperament, the affected infants tended to respond more intensely with a higher number of vocalizations per minute. The affected infants tended to show a marked inability to quiet themselves when left alone or when picked up and cuddled.

Beginning at 2 months of age, affected infants and the controls were tested using the Modified Bayley test (Champoux et al., 1990, Am. J. Primatol. 22:61-67). The Modified Bayley Scale includes three subtests: cognitive, motor, and behavior. The cognitive subtest contains problem-solving items examining sensory-perceptual acuities, discriminations, and the ability to respond to these. After 2 months of age, the affected infants scored lower on the motor subtest as compared to the normal monkeys at all other testing time points. On the items that measure behavior/social orientation, the most marked difference at all time points between affected and normal monkeys was irritability level. These two neurobehavioral assessment tools detected differences between affected and normal monkeys.

Neurophysiology: Serial nerve conduction studies beginning within the first 2 months of life in 4 homozygous, 2 heterozygous and 2 normal rhesus monkeys (Macaca mulatta) to characterize the peripheral neuropathy were performed. Because there were no significant differences between the carrier and normal groups, these groups were combined to create the unaffected comparison group for all subsequent analyses. For each nerve, a significant interaction effect of group by age was found (Median nerve: F(1,57)=24.06, p<0.0001; Tibial nerve: F(1,58)=26.44, p<0.0001; Ulnar nerve: F(1,59=28.68, p<0.0001). Mean conduction velocities of the median, ulnar, and tibial nerves were significantly slower in the affected than unaffected monkeys at all ages (P<0.0001) (FIG. 7, Weimer et al., 2005, Muscle Nerve 32(2): 185-190). In the affected monkeys, bilateral median, ulnar, and tibial motor nerve conduction studies showed normal compound muscle action potential amplitudes, but all nerves exhibited severe prolongation of distal latencies and severe slowing of conduction velocities. F-waves were well formed and reproducible, but latencies were severely prolonged in affected monkeys. The conduction velocity differences became more apparent between the affected and unaffected as the monkeys aged. There was no evidence of excessive temporal dispersion in any nerve. In the affected monkeys, the degree of conduction slowing was remarkably uniform along all segments of each nerve and highly concordant between nerves. When compared to the unaffected monkeys, the serial conduction velocities suggested occurrence of dysmyelination followed by demyelination in the affected monkeys. These findings are diagnostic of a severe primarily demyelinating polyneuropathy and are in accordance with the expected electrophysiological phenotype of GLD. The diffuse and uniform slowing of motor nerve conduction velocities are typical for an inherited demyelinating (hypomyelinating) neuropathy secondary to a defect in myelination of the peripheral nervous system. Similar studies done on the carrier monkeys were within normal limits for rhesus monkeys.

Neuroimaging: All affected infants along with age-matched carrier and non-carrier controls were studied monthly using MRI. The MRI sequences included a saggital T1-weighted scan, saggital T2-weighted scan, axial T1-weighted scan, axial proton density, axial T2-weighted scan, coronal T2-weighted scan, and postcontrast axial and coronal T1-weighted scans. When needed, Prohance (Gadoteridol) was administered at 0.1 mmole/kg intravenously for contrast. The MRI interpretation included the location, extent, and morphology of white matter disease, as well as associated intracranial cortical and ventricular changes.

The data from early MR imaging analysis indicated that no definite anomalies were observed. The affected animals showed no abnormal changes in either their grey or white matter, when compared to age matched controls. In addition, the ventricle system and cortical sulci were unremarkable, as well. It is important to note that the animals that have been thoroughly analyzed had to be necropsied at approximately 100 days of life. It is plausible that changes in the CNS occur later in life. In support of this theory, one Krabbe animal that lived over 22 months was noted to have increased areas of T2 signal associated with the trigonal area of the posterior horns of the lateral ventricles. In humans, this is often described as an early finding of Krabbe disease.

Pathology: Upon pathological examination, all euthanized monkeys (n=7) had globoid cells in the white matter of the CNS. Peripheral nerves were enlarged and firm. The cerebral, cerebellar, and spinal cord white tracts were heavily infiltrated with PAS-positive multinucleated globoid cells and smaller PAS-positive macrophages. All had severe, diffuse demyelination in the cerebrum and nerves. Fibers of the peripheral nerves were widely separated and the intervening space contained loose fibrillar fibrous connective tissue and finely granular to homogeneous eosinophilic material. There were no apparent myelin sheaths around nerve fibers, which result in loss of normal CNS architecture. Many of the animals had mild to moderate inflammation in the lungs. Two of the females also had acute cervix/uterine inflammation. In the single stillborn infant, mild demyelinating lesions were apparent in the CNS and peripheral nerves and globoid cells were present in the CNS.

### Example 5

### Transplantation of ASCs—In Vitro and In Vivo Analysis

Autologous transplants of ASCs will be performed in both canines and primates.

The materials and methods used in the experiments presented in this Example are now described.

In vitro Analyses: All studies are conducted with a minimum of five canine donors and five primate donors for each gender. Thus, a minimum of ten canine and ten rhesus specimens are processed.

ASC Isolation: Subcutaneous adipose tissue is harvested from Cairn Terriers (LSU—School of Veterinary Medicine) or rhesus monkeys (Tulane Primate Center) in accordance with a surgical protocol reviewed and approved by the Institutional Animal Care and Use Committees. The canine and rhesus subcutaneous adipose tissue are processed for the isolation of ASCs in an identical manner to that developed for human ASC isolation from lipoaspirates (Dubois et al., 2005, Adipocytes 1(3):139-144). All tissue processing, isolation, and culture is conducted with screened serum lots selected for their ability to support the proliferation and adipogenic differentiation of huASCs. Likewise, the tissue culture medium, plasticware, and other reagents are standardized across species to remove any potential sources of deviation or artifact in the production process.

Tissue is minced, digested with type I collagenase at 37° C., and separated by differential centrifugation at room temperature. The pelleted stromal vascular fraction (SVF) is seeded at constant density of 0.156 gm tissue digest per cm² surface area during the initial plating, and at densities of 500 or 5,000 cells per cm² surface area during subsequent passages. Additional plating conditions are outlined below for specific assays. Cell yield per unit weight of tissue is determined. In addition, cell proliferation rates in culture are calculated for each stage of passage. When necessary, cells are cryopreserved according to parameters optimized for the huASCs (Thirumala et al., 2005, September-October; 21(5):1511-24). These steps ensure that the “manufacturing” procedure for canine ASCs (caASCs) and primate ASCs (pASCs) does not deviate from that of huASCs. These steps also simplify the analysis of any cross species comparison of the ASC properties in future steps.

Colony Forming Unit Assays: The frequency of colony forming units for specific lineages or phenotypes are determined by limit dilution based on the Poisson distribution (Mitchell et al., Stem Cells online Jan. 12, 2006: 2005-0235). The nucleated cell density in the stroma-vascular fraction (SVF) is determined. Beginning at a density of 10⁴ cells/well, 2-fold serial dilutions of nucleated SVF cells are seeded on a 96 well plate and maintained for a period of 9 days. At this time, plates are harvested directly for staining with toluidine blue (CFU-Fibroblast assay) or alkaline phosphatase (CFU-ALP assay). Additional plates are induced with adipogenic or osteogenic differentiation medium and maintained in culture for an additional 9 or 21 days prior to histochemical staining for Oil Red O (CFU-Adipocyte assay) or Alizarin Red (CFU-Osteoblast assay). The presence or absence of cell colonies in the wells at each cell density are recorded and the lineage specific CFU frequency calculated. These analyses determine the CFU frequency in the SVF. Comparable studies are performed with the ASCs following passage 2 or passage 4 to determine if any enrichment or loss of particular lineages occurs following the adhesion and expansion process.

Differentiation Potential: The differentiation potential of prASC at passages 2, 4, and 6 of expansion is assessed for the following lineage pathways: adipogenic, chondrogenic, neuronal, and osteogenic using published protocols and detection methods (Guilak et al., 2006, Journal of Cellular Physiology, January; 206(1):229-37). Differentiation is determined based on morphology, histochemical and/or immunohistochemical staining, and PCR detection of lineage specific gene markers (Halvorsen et al., 2001, Metabolism 50(4):407-413; Halvorsen et al., 2001, Tissue Eng. 7(6):729-741; Safford et al., 2002, Biochem. Biophys. Res. Commun. 294(2):371-379; Safford et al., 2004, Exp. Neurol. 187(2):319-328; Sen et al., 2001, J. Cell Biochem. 81(2):312-319; Wickham et al., 2003, Clin. Orthop. (412):196-212; Erickson et al., 2002, Biochem. Biophys. Res. Commun. 290(2):763-769; Guilak et al., Journal of Cellular Physiology, In Press).

Galactocerebrosidase Activity: Biochemical assays of galactocerebrosidase enzyme activity are performed with the undifferentiated prASCs and huASCs as a function of passage. Levels of enzyme activity is compared to that detected in peripheral blood mononuclear cells obtained from primates and humans.

In vivo Analyses: Intravenous Transplantation Protocol: During culture, the ASCs are labeled by incubation with bromodeoxyuridine (BrdU) for tracking purposes. The ASC at passage 2 or passage 6 are harvested by trypsin/EDTA digestion, washed, and suspended in room temperature serum free medium at a concentration of no more than 10⁶ cells per ml. Cytologenetic testing/chromosome spreads are performed on aliquots of the prASCs to document any evidence of aneuploidy. Cells are administered to immunodeficient mice (NOD/SCID) by tail vein injection at doses of up to 4×10⁷ cells/kg body weight or approximately 10⁶ cells per animal. Control cohorts of mice are injected with an equal volume of media alone. Following periods of 2, 8, or 26 weeks, animals are sacrificed and necropsies are performed to determine evidence of tumor formation. Immunohistology is performed on serial sections of major tissues (brain, liver, kidney, heart, lungs, adipose tissue) fixed in formalin fixation and paraffin embedded using antirdU antibodies to detect the migration and survival of transplanted, labeled ASCs.

Flow Cytometry: All studies are performed on prASCs isolated from a minimum of 5 donors of each gender. Flow cytometric analyses are performed on rhesus adipose tissue derived cells at various stages of isolation and expansion; from SVF to Passage 1 through 4. Cells are fixed and stained with a panel of antibodies (see below) according to published procedures; further antigens may be included. As needed, huASCs serve as controls. Rhesus peripheral blood mononuclear cells serve as positive controls for hematopoietic and other antigens that are expected to be absent on the surface of prASCs. Initial analyses are performed on both fresh and cryopreserved prASCs. If it is determined that the outcomes are unchanged by cryopreservation, further data collection on cryopreserved materials is to be continued to increase the flexibility of the experiments and to reduce flow cytometry costs. In the event that cryopreservation alters the surface immunophenotype significantly, data is to be collected on fresh prASCs only. Additional studies to examine the expression level of ALDH in the rhesus ASCs at the various stages of isolation and expansion are to be performed. Studies employ a commercially available kit of reagents from StemCo (Durham, N.C.) according to published methods (Mitchell et al., Stem Cells online Jan. 12, 2006: 2005-0235). The ability to inhibit enzyme activity with the substrate analog, DEAB, serves as a negative control, while ALDH expression by huASCs serves as a positive control.

Proteomics: All studies are performed on prASCs isolated from a minimum of 5 donors of each gender. Initial studies are conducted using Passage 1 prASCs in order to match the existing data set obtained for huASCs (Delany et al., 2005, Mol. Cell Proteomics 4:731-7). Duplicate 2D gels are performed on protein extracts from each donor and a total of 10 gels will be examined. A “master” gel is generated for each gender and for both genders. Up to 200 features/spots from the “master” gel of both genders are selected for mass spectroscopic analysis and protein identification. The prASC “master” gels are compared directly to the annotated “master” gel prepared for the huASCs. From this analysis, the percentage of protein features conserved between species is determined. Features/spots unique to either the female or male prASC “master” gels by mass spectroscopy are identified.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

The disclosures of each and every patent, patent application, and publication cited herein are hereby incorporated herein by reference in their entirety.

1. A method of treating at least one symptom of a leukodystrophy in a mammal, said method comprising administering to said mammal an isolated adipose-derived stem cell (ASC) exhibiting a non-immunogenic characteristic.

2. The method of claim 1, wherein said leukodystrophy is selected from the group consisting of Krabbe disease, adrenoleukodystrophy/adrenomyeloneuropathy, Aicardi-Goutieres syndrome, Alexanders disease, childhood ataxia with diffuse central nervous system hypomyelination (CACH), cerebral autosomal dominant arteriopathy with subcortical infarcts and leukoencephalopathy (CADASIL), Canavan disease, cerebrotendinous xanthomatosis, metachromatic leukodystrophy, neonatal adrenoleukodystrophy, ovarioleukodystrophy syndrome, Pelizaeus-Merzbacher disease, Refsum disease, Van der Knaap syndrome and Zellweger syndrome.

3. The method of claim 2, wherein said leukodystrophy is Krabbe disease.

4. The method of claim 1, wherein said non-immunogenic characteristic is galactocerebrosidase expression.

5. The method of claim 1, wherein said ASC is differentiated into a cell that expresses galactocerebrosidase.

6. The method of claim 4, wherein said galactocerebrosidase is expressed in an amount effective to reduce a level of psychosine in white matter of a brain of said mammal.

7. The method of claim 5, wherein said galactocerebrosidase is expressed in an amount effective to reduce a level of psychosine in white matter of a brain of said mammal.

8. The method of claim 1, wherein said at least one symptom is selected from the group consisting of axonal degeneration, fibrosis, macrophage infiltration, astrocytosis, decrease in myelin, irritability, excessive crying, loss of motor skills, hypersensitivity to external stimuli, stiffness of muscles, extension of arms and legs, clenched fingers, hypotonicity, blindness and deafness.

9. The method of claim 1, wherein said ASC is administered intravenously to said mammal.

10. The method of claim 1, wherein said ASC is selected from the group consisting of allogeneic and autologous with respect to said mammal.

11. The method of claim 1, wherein said ASC further comprises a biocompatible matrix.

12. The method of claim 11, wherein said biocompatible matrix is selected from the group consisting of calcium alginate, agarose, fibrin, collagen, laminin, fibronectin, glycosaminoglycan, hyaluronic acid, heparin sulfate, chondroitin sulfate A, dermatan sulfate, and bone matrix gelatin.

13. The method of claim 1, wherein said mammal is a primate.

14. The method of claim 13, wherein said primate is selected from the group consisting of a human and monkey.

15. The method of claim 13, wherein said primate is human.

16. The method of claim 1, wherein said ASC is cultured in vitro for a period of time without being induced to differentiate prior to said administration of said cell to said mammal.

17. A method of identifying an ASC that expresses galactocerebrosidase in a population of cells derived from adipose tissue, said method comprising providing a substrate specific for galactocerebrosidase to said population of cells, wherein said substrate is degraded when said galactocerebrosidase is present in said ASC thereby identifying said ASC in said population of cells.

18. The method of claim 17, wherein said substrate is galactosylsphingosine or galactosylceramide.

19. The method of claim 17, wherein said ASC is differentiated into a cell exhibiting at least one characteristic of a cell selected from the group consisting of a leukocyte, a fibroblast, a chondrocyte, an osteoblast, a Schwann cell, an oligodendrocyte and a neuron.

20. A method of increasing a level of galactocerebrosidase in a tissue or mammal, said method comprising administering to said mammal an isolated ASC exhibiting a non-immunogenic characteristic, wherein said ASC differentiates in vivo or in vitro into a cell that expresses galactocerebrosidase.

21. The method of claim 20, wherein said mammal is a primate.

22. The method of claim 21, wherein said primate is selected from the group consisting of a human and monkey.

23. The method of claim 21, wherein said primate is a human.

24. The method of claim 20, wherein said differentiated ASC is a cell exhibiting at least one characteristic of a cell selected from the group consisting of a leukocyte, a fibroblast, a chondrocyte, an osteoblast, a Schwann cell, an oligodendrocyte and a neuron.

25. The method of claim 20, wherein said ASC is cultured in vitro for a period of time without being induced to differentiate prior to said administration of said cell to said mammal.

26. The method of claim 20, wherein said ASC is allogeneic with respect to said mammal.

27. The method of claim 20, wherein said ASC is autologous with respect to said mammal.

28. The method of claim 20, wherein said ASC further comprises a biocompatible matrix.

29. The method of claim 28, wherein said biocompatible matrix is selected from the group consisting of calcium alginate, agarose, fibrin, collagen, laminin, fibronectin, glycosaminoglycan, hyaluronic acid, heparin sulfate, chondroitin sulfate A, dermatan sulfate, and bone matrix gelatin.

30. An isolated ASC exhibiting a non-immunogenic characteristic, wherein said ASC expresses galactocerebrosidase and is identified by providing a substrate specific for galactocerebrosidase to said population of cells, wherein said substrate is degraded when said galactocerebrosidase is present in said ASC thereby identifying said ASC in said population of cells.

31. The isolated ASC of claim 30, wherein said ASC is isolated from a primate.

32. The isolated ASC of claim 30, wherein said primate is selected from the group consisting of a human and monkey.

33. The isolated ASC of claim 31, wherein said primate is a human.

34. The isolated ASC of claim 30, wherein said ASC is differentiated into a cell selected from the group consisting of a leukocyte, a fibroblast, a chondrocyte, an osteoblast, a Schwann cell, an oligodendrocyte and a neuron.

35. The isolated ASC of claim 30, wherein said ASC is allogeneic respect to a recipient thereof.

36. The isolated ASC of claim 30, wherein said ASC is autologous with respect to a recipient thereof.

37. A substantially homogeneous population of isolated ASCs, wherein said isolated ASC exhibits a non-immunogenic characteristic, wherein said ASC expresses galactocerebrosidase and is identified by providing a substrate specific for galactocerebrosidase to said population of cells, wherein said substrate is degraded when said galactocerebrosidase is present in said ASC thereby identifying said ASC in said population of cells.

38. An isolated ASC genetically modified to express galactocerebrosidase.

39. The isolated ASC of claim 38, wherein said galactocerebrosidase is from a human, monkey, mouse or rat.

40. The isolated ASC of claim 38, wherein said ASC is transfected with a vector expressing galactocerebrosidase.

41. The isolated ASC of claim 38, wherein said ASC is a human cell.
